(19) United States
(12) Patent Application Publication
(10) Pub. No.: US 2023/0392158 A1
(43) Pub. Date: Dec. 7, 2023

(54) CONTROLLING CELLULAR BEHAVIOR USING FEED-FORWARD CIRCUITS

(71) Applicant: The Regents of the University of California, Oakland, CA (US)

(72) Inventors: Alexandra WESTBROOK, San Francisco, CA (US); Andrew H. NG, San Francisco, CA (US); Hana EL-SAMAD, San Francisco, CA (US)

(21) Appl. No.: 18/034,045

(22) PCT Filed: Jan. 11, 2022
(86) PCT No.: PCT/US2022/012033

§ 371 Date: Apr. 26, 2023

Related U.S. Application Data
(60) Provisional application No. 63/136,560, filed on Jan. 12, 2021.

(51) Int. Cl. C12N 15/63; C12N 5/078

(52) U.S. Cl. CPC C12N 15/635; C12N 5/0634

(57) ABSTRACT

Provided herein are a variety of molecular feed-forward circuits. In some embodiments a molecular feed-forward circuit may comprise a first nucleic acid comprising a first nucleotide sequence encoding a regulatory protein that is activated by a first exogenous stimulus, and a second nucleic acid comprising a second nucleotide sequence encoding a signaling protein that is activated by the first exogenous stimulus, where the target protein has a regulatory motif and the activated regulatory protein inactivates the target protein via the regulatory motif. The molecular circuit can further comprise a controller protein that is activated by a second exogenous signal, wherein the controller protein controls the interaction between the regulatory protein and the target protein. In this circuit activation of the regulatory protein is delayed relative to the activation of the target protein. The system can be used to produce a pulse of signaling and/or a concentration filter, for example.

### CROSS-REFERENCING

This application claims the benefit of U.S. provisional application Ser. No. 63/136,560, filed on Jan. 12, 2021, which application is incorporated herein in its entirety for all purposes.

### GOVERNMENT RIGHTS

This invention was made with government support under grant no. HR0011-16-2-0045 awarded by The Defense Advanced Research Projects Agency. The government has certain rights in the invention.

### BACKGROUND

There has been significant progress in the construction and characterization of engineered regulatory molecules for precise control of gene expression. Synthetic biologists have built and optimized an arsenal of genetic switches that affect all aspects of transcription, translation, and degradation. These regulatory parts have been wired into small ‘circuits’ to expand the range of functionalities beyond simple activation and repression to behaviors such as: bistability, oscillation, buffering, logic gates, amplification, feedback, feedforward, and more. However, most circuits have been single entities built from hijacked endogenous parts with little room for modularity and orthogonality. In order to create more complex networks, these parts need to be modular and composable, performing their function within the network with minimal undesired interactions.

### SUMMARY

Provided herein are a variety of molecular feed-forward circuits where, in many embodiments, the circuit may comprise: (a) a nucleic acid encoding a target protein that comprises a regulatory motif that is not native to the target protein, (b) a nucleic acid encoding an inactivating protein, wherein the inactivating protein binds to the regulatory motif and inactivates the target protein; and (c) an actuating protein that, in response to a first external stimulus, independently activates expression the target protein of (a) and the inactivating protein of (b).

In certain embodiments of this circuit, the activation of the inactivating protein is delayed relative to the activation of the target protein. Particularly, in response to the stimulus, the cell produces a target protein and, after a delay, the cell produces an inactivating protein that inhibits the target protein thereby inhibiting the cell's response to the stimulus. Because of the initial activation of the target protein and later activation of the inactivating protein, the cell has the ability to respond to the stimulus over a short period of time after which the response subsides, thereby producing a pulse of a response to the stimulus.

In certain other embodiments, the circuit comprises an inactivating protein and a target protein, both activated by a first exogenous stimulus, and a controller protein that is activated by a second exogenous stimulus, wherein the controller protein controls the interactions between the inactivating protein and the target protein. The cell produces: 1) a target protein and an inactivating protein in response to the first exogenous stimulus and 2) a controller protein in response to the second exogenous stimulus. The controller protein relieves the inactivating protein mediated inhibition of the target protein, thereby allowing the cell to activate the target protein in response to the first stimulus. When the strength of the first stimulus exceeds a threshold, the activation of the inactivating protein is sufficiently strong so that the controller protein cannot relieve the inactivating protein mediated inhibition of the target protein and the inactivating protein inhibits the target protein. Thus, the cell does not activate the target protein in response to the first stimulus when the strength of the first stimulus exceeds the threshold. Thus, the relative strengths of the first and the second stimuli and the cell's response to these stimuli provide the cell the ability to respond to the first exogenous stimulus only up to a threshold strength of the first exogenous stimulus.

This system can be used to control signaling by the target protein. For example, the system can be used to produce a pulse of signaling and/or a concentration filter, for example. Examples of such systems are described in greater detail below.

The cells and the methods disclosed herein provide molecular circuits, particularly, molecular circuits, such as pulse generation and concentration filters that provide control of cellular response to exogenous stimuli.

For example, in many embodiments, a circuit can be thought of as a “pulse generator” in that it can control a cell's response to an exogenous stimulus such that the cell responds to the stimulus for a brief period (e.g., less than a minute, less than 30 minutes or less than an hour, less than 12 hr, etc.) and the response then subsides, thereby generating a “pulse” of response, as illustrated in FIG. 2. In other embodiments, a circuit can be thought of as a concentration filter, where a “concentration filter” controls a cell's response to an exogenous stimulus that is within a range. In these embodiments, the filter can be designed to have positive feedback, which also filters out low concentrations. As such, in these embodiments, a response may be produced only in a particular band of concentrations.

A pulse generator molecular circuit comprises an inactivating protein that is activated by a first exogenous stimulus and a target protein that is activated by the first exogenous stimulus, wherein the target protein has a regulatory motif and the activated inactivating protein inactivates the target protein via the regulatory motif. Also, the activation of the inactivating protein is delayed relative to the activation of the target protein.

A concentration filter molecular circuit comprises, in addition to the pulse generator molecular circuit, a controller protein that controls the interaction between the inactivating protein and the target protein.

Also provided are nucleic acids encoding molecular circuits and cells containing such nucleic acids. A cell containing such molecular circuits can be genetically modified to contain one or more nucleic acids encoding the inactivating protein, the target protein, and, when present, the controller protein. Such nucleic acids can contain nucleotide sequences encoding the corresponding proteins under the control of appropriate regulatory sequences, such as promoters.

Methods of using the molecular circuits are also provided, including e.g., methods of modulating a signaling pathway of a cell that include genetically modifying the cell with such molecular circuits. Further provided are methods of using the cells comprising molecular circuits disclosed herein to treat a disease in a subject.

### BRIEF DESCRIPTION OF THE DRAWINGS

This patent or application file contains at least one drawing executed in color. Copies of this patent or patent application publication, with color drawing(s), will be provided by the Office upon request and payment of the necessary fee.

FIG. 1. Building a signal processing toolbox with de-novo designed proteins.

FIG. 2. Signal processing circuits can be used to improve specificity in cell therapies.

FIG. 3. The pulse generator could act as a delayed off switch to reduce off target effects.

FIG. 4. The pulse generator could act as a delayed off switch to reduce off target effects.

FIG. 5. The pulse generator could act as a delayed off switch to reduce off target effects.

FIG. 6. The filter could be used to distinguish between cells of varying antigen expressions.

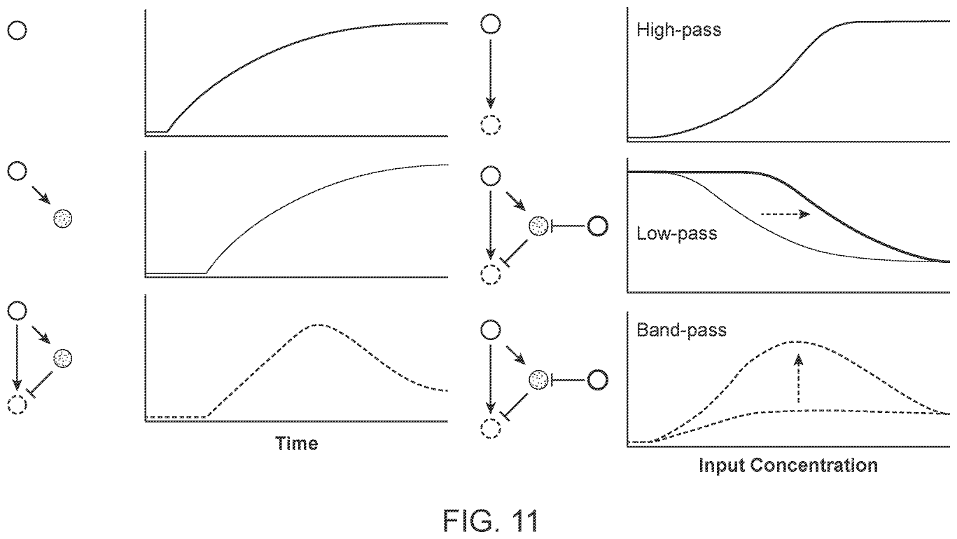

FIG. 7. The filter could be used to distinguish between cells of varying antigen expressions.

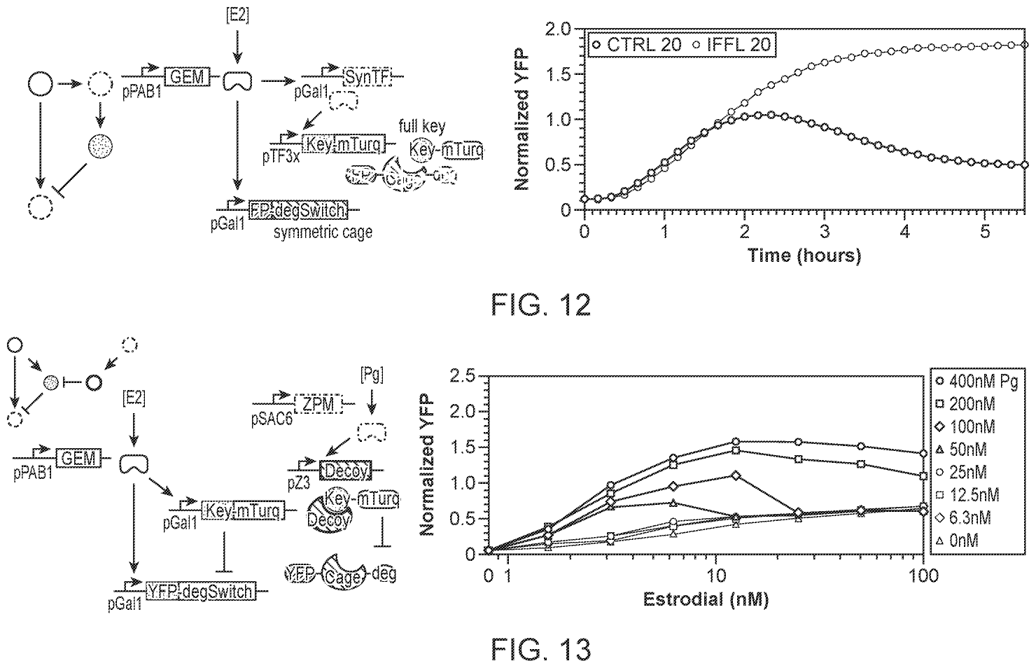

FIG. 8. The filter could be used to distinguish between cells of varying antigen expressions.

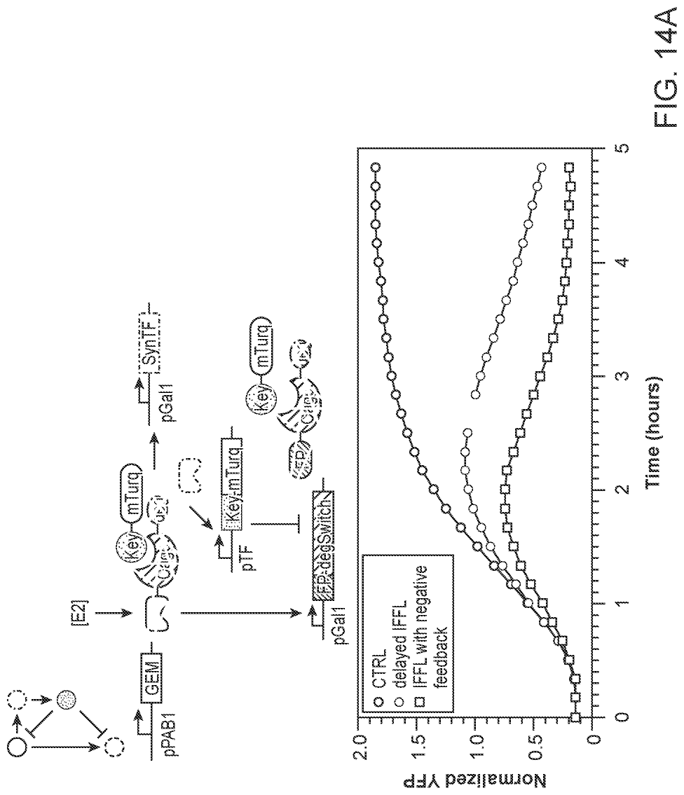

FIG. 9. The filter could be used to distinguish between cells of varying antigen expressions.

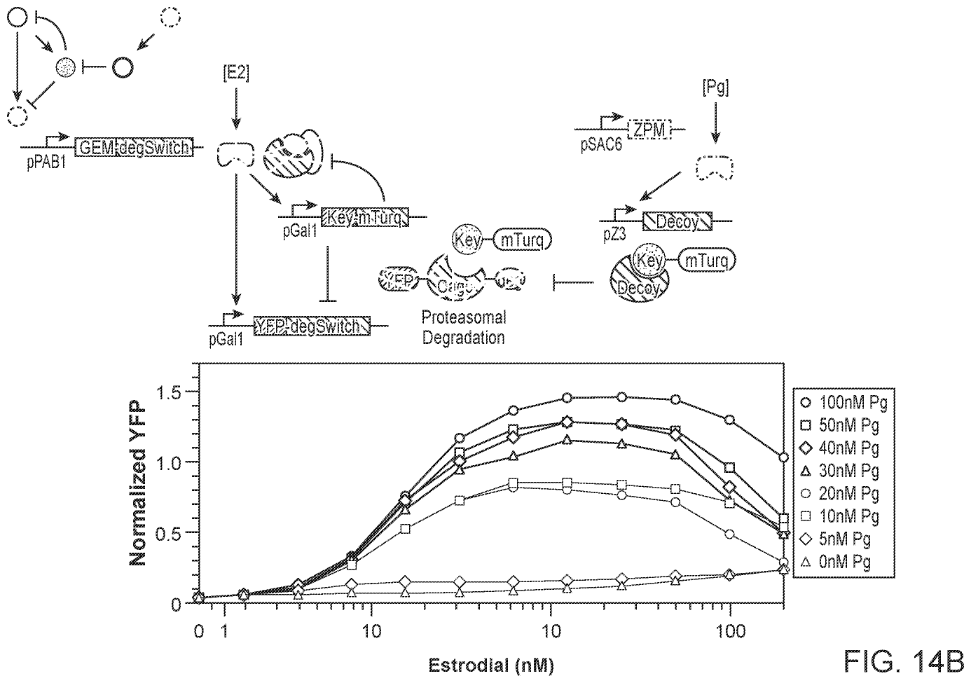

FIG. 10. degronLOCKR is degraded in the presence of the key.

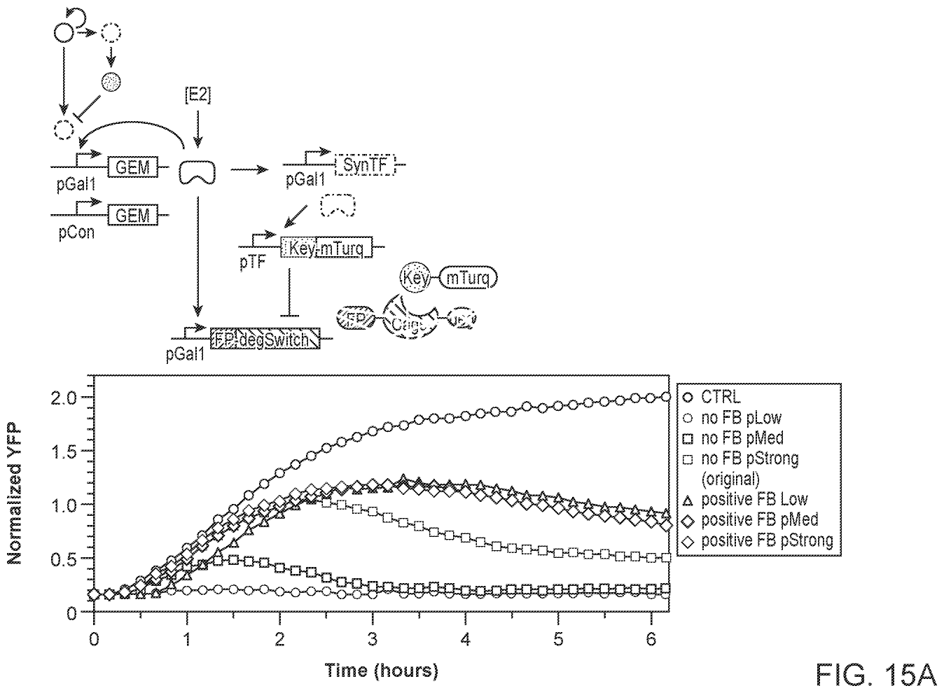

FIG. 11. An I1-FFL is a base design for non-monotonic signal processing units in both time (pulse generation) and steady state (concentration filter). I1-FFLs have been used to implement band-pass filters, fold-change detection, biosensing, and noise buffering. An I1-FFL comprises of an activator X that activates a gene Z and simultaneously its repressor, Y. Activation is triggered immediately by X, while the dominating repression occurs with a delay due to the presence of the intermediate component Y, producing a pulse of gene expression.

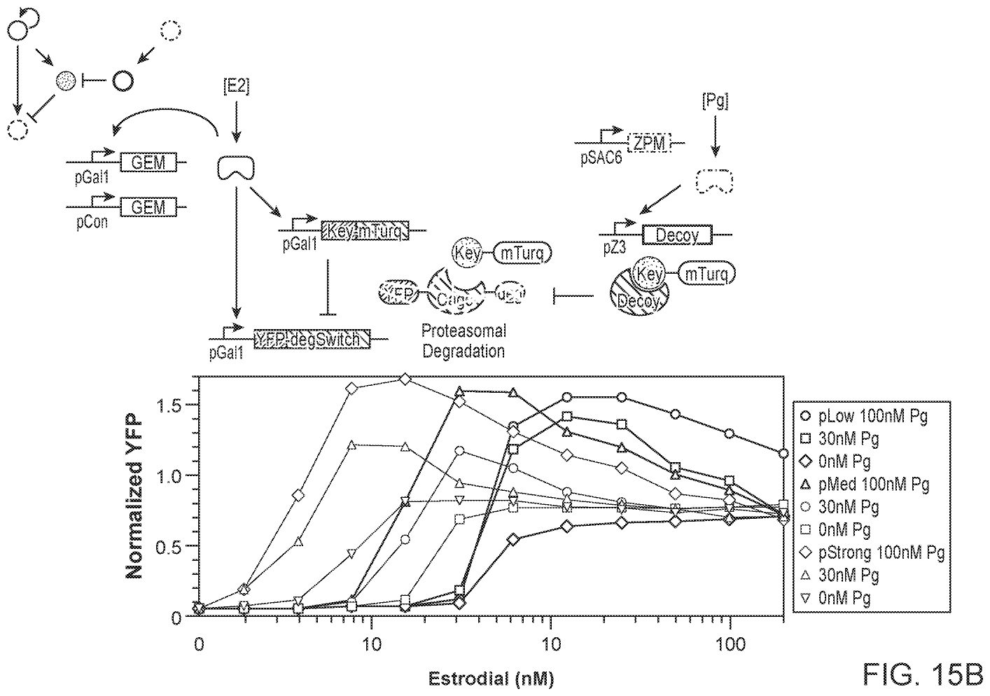

An I1-FFL can also be tuned to create a filter that only activates high expression of output at intermediate levels of input. The threshold of intermediate Y repression can be tuned using a sequestration component that binds to Y to sequester it and stop it from repressing. This creates a threshold that Y must overcome to significantly repress.

FIG. 12. I1-FFL with a transcriptional delay produces a pulse of gene expression. The delayed I1-FFL uses estradiol as an input that activates a transcription factor, GEM. GEM activates gene expression of a synthetic transcription factor (synTF) and YFP-degSwitch. SynTF activates expression of key, causing a delay. YFP-degSwitch degradation is activated by the binding of the key to the degSwitch. Data was collected after inducing with 20 nM of E2 at t=0 using automated flow cytometry over 5.5 hours.

FIG. 13. I1-FFL with a decoy produces a filter. The three node I1-FFL (without delay) and the decoy were combined to create a tunable key threshold. The transcription factor, ZPM, is used to dial the expression of decoy to a level that provides filter behavior (˜100 nM). The filter only produces high YFP for intermediate levels of estradiol induction. Data was collected after 5 hours of progesterone and estradiol induction using flow cytometry.

FIGS. 14A-14B. Pulse generator (14A) and concentration filter (14B) circuits with negative feedback mechanisms. Negative feedback reduces the steady state expression. Negative feedback was added on GEM by fusing a degSwitch to GEM. When the key is expressed, it also represses GEM, reducing the steady state in both the pulse generator and the filter.

FIGS. 15A-15B. Pulse generator (15A) and concentration filter (15B) circuits with positive feedback mechanisms. Positive feedback shifts the threshold of activation in the filter and lengthens the pulse. Positive feedback was added on GEM by adding another construct—GEM produced by its own promoter pGal. GEM is constitutively expressed at low, medium, or strong levels. When the constitutive GEM is activated by E2, it activates the circuit and produces itself in positive feedback. This amplifies and increases the sensitivity of the response. Additionally, tuning the strength of the constitutive promoter shifts the filter response.

FIGS. 16A-16B. Pulse generator (16A) and concentration filter (16B) circuits with both positive and negative feedback mechanisms. Combining positive and negative feedback in the IFFL and filter. Positive and negative feedback were combined to get a tunable and sensitive response that also has lower steady state expression. The positive feedback was observed to be stronger than the negative feedback. This relative strength of the positive and negative feedbacks can be tuned.

FIGS. 17A and 17B. A Schematics of the designer proteins and transcription factors. Two inducible transcription factors and one non-inducible transcription factor is used to induce expression of the designer proteins, create transcriptional delays, and implement positive feedback. Three designer proteins act post-transcriptionally to repress one another. B Schematics of circuits that can be constructed using parts described in (A) including: tunable degradation, positive and negative feedback, sequestration, 3-node I1-FFL, pulse generator, and filter. Each box represents a single circuit. A small model graph shows the expected output from each circuit.

FIGS. 18A-18C Gene expression data for small circuits. (A) The positive feedback network amplifies and increases sensitivity. Positive feedback on GEM is constructed by adding another GEM under control of pGall. Constitutive GEM expression controls the strength of the positive feedback. (B) Sequestration shifts the cage/key transfer function. The decoy binds to the key, creating a threshold the key must overcome to bind and degrade the cage. (C) The 3-node I1-FFL produces a small pulse. The 3-node I1-FFI uses GEM as the input node, key as the intermediate node, and YFP-cage as the output node. GEM activates key and YFP-cage and then key activates degradation of YFP-cage.

FIGS. 19A-19L. Schematics and results for the pulse generators. (A) Schematic for the 4-node I1-FFL. A transcriptional delay was added to the 3-node I1-FFL using a SynTF to create the 4-node I1-FFL. GEM activates SynTF which activates the key. The control circuit (CTRL) was GEM activating YFP (schematic in box). (B) Time course data for the 4-node I1-FFL. YFP fluorescence was measured every 10 minutes by automated flow cytometry. Cultures were induced with 1.25, 2.5, 5, or 10 nM of estradiol for both the control (CTRL) and 4-node I1-FFL. The circles represent the average of biological triplicate and the shaded region represents one standard deviation. (C) Fittings for the 4-node I1-FFL. The circles represent the average experimental data at each time point. The shaded region represents the 2.5 and 97.5 percentile of fitting. (D) Schematic for the 4-node I1-FFL with negative feedback. Negative feedback is added into the pulse generator by fusing a cage to GEM. When the key is expressed it now degrades both the output, YFP, and the input, GEM, to produce a lower steady state value. The control circuit (CTRL) was GEM-cage activating YFP. (E) Time course data for the 4-node I1-FFL with negative feedback. YFP fluorescence was measured every 10 minutes by automated flow cytometry. Cultures were induced with 1.25, 2.5, 5, or 10 nM of estradiol for both the control (CTRL) and 4-node I1-FFL with negative feedback. The circles represent the average of biological triplicate and the shaded region represents one standard deviation. (F) Fittings for the 4-node I1-FFL with negative feedback. The circles represent the average experimental data at each time point. The shaded region represents the 2.5 and 97.5 percentile of fitting. (G) Schematic for the 4-node I1-FFL with positive feedback. Positive feedback was added onto the input node, GEM, to amplify and lengthen the pulse. The control circuit (CTRL) was GEM with positive feedback activating YFP. (H) Time course data for the 4-node I1-FFL with positive feedback. YFP fluorescence was measured every 10 minutes by automated flow cytometry. Cultures were induced with 10 nM of estradiol for both the control (CTRL) and 4-node I1-FFL with positive feedback. The circles represent the average of biological triplicate and the shaded region represents one standard deviation. (I) Fittings for the 4-node I1-FFL with positive feedback. The circles represent the average experimental data at each time point. The shaded region represents the 2.5 and 97.5 percentile of fitting. (J) Schematic for the 4-node I1-FFL with positive and negative feedback. Positive and negative feedback were combined to produce a longer pulse with lower steady state expression. The control circuit (CTRL) was GEM-cage with positive feedback activating YFP. (K) Time course data for the 4-node I1-FFL with positive and negative feedback. YFP fluorescence was measured every 10 minutes by automated flow cytometry. Cultures were induced with 2.5 or 10 nM of estradiol for both the control (CTRL) and 4-node I1-FFL with positive and negative feedback. The circles represent the average of biological triplicate and the shaded region represents one standard deviation. (L) Fittings for the 4-node I1-FFL with positive and negative feedback. The circles represent the average experimental data at each time point. The shaded region represents the 2.5 and 97.5 percentile of fitting.

FIGS. 20A-20D Schematics and experimental results for the filters. (A) The basic filter design and data. The filter uses the 3-node IFFL combined with sequestration to tune the key repression and produce steady state non-monotonic behaviour. (B) Adding negative feedback into the pulse reduces expression at high estradiol concentrations. Negative feedback is added into the filter by fusing a cage to GEM. When key is highly expressed and overcomes the decoy threshold it now degrades the output, YFP, and the input, GEM to produce a lower steady state value. (C) Adding positive feedback into the filter shifts the filter peak. Positive feedback was added into the input node of the filter, GEM. This allowed the shifting of the peak by shifting the strength of positive feedback. (D) Combining positive and negative feedback.

FIG. 21 schematically illustrates an example of the present fusion protein.

FIG. 22 schematically illustrates various models for cullin-RING E3 ligases. These complexes promote the transfer of ubiquitin from the E2 to the substrate, which targets the protein for degradation. Many complexes contain an adapter protein (e.g., SKP1 for CUL1 and CUL7, Elongin B/C for CUL2 and CUL5, BTB for CUL3 and DDB1 for CUL4A/b) as well as a receptor protein (F-box proteins for CUL1, VHL-box proteins for CUL2, DCAFs for CUL4A and 4B, SOCS for CUL5 and FbxW8 for CUL7) and a RING protein (RB1/2).

FIG. 23: Lysine to arginine substitution significantly improves STUD activity. Either a GFP nanobody (vhhGFP4) or SynZIP (SZ18) were used to target a GFP (or in the case of the SZ18 STUD, GFP-SZ17. SZ17 and SZ18 form a cognate pair). GFP % Degradation was measured compared to GFP fluorescence in the absence of the STUD.

FIG. 24: MG132 proteasome inhibitor confirms the effect of STUD is mediated by the proteasome. Primary human CD4+ T cells expressing different variants of the GFP nanobody STUD were few 5 uM MG132 and fluorescence was measured at 1 and 3 hours post induction. The mutant nanobody was the only experimental group that exhibited an increase in fluorescence over time, suggesting the effect of the STUD is mediated by protein degradation through the proteasome.

FIG. 25: Optimizing STUD activity via linker modification in Jurkat cells. A variety of flexible (GS) and rigid linkers were tested between the SynZIP targeting domain and degron on the STUD. It was observed that flexible linkers generally outperformed rigid linkers, and in particular the 5×GS linker produced the greatest degradation

FIG. 26: Design of a circuit to test STUD induced degradation of a synthetic transcription factor. VPR-NS3-ZF3 drives activation of the pZF3(8x)_ybTATA promoter in response to induction with GRZ. Three different circuit configurations were explored. Feedback, where STUD is driven off the pZF3 promoter, GFP alone, where no STUD is expressed, and Constitutive STUD, where the STUD is expressed off the pPGK promoter

FIG. 27: ZF3 circuit dose responses demonstrate the functionality of the soluble STUD to degrade a transcription factor. The circuits were transduced into Jurkat cells and induced with a range of GRZ concentrations to activate the TF. GFP fluorescence was measured 72 hours later

### DEFINITIONS

The terms “synthetic,” “chimeric,” and “engineered” as used herein generally refer to artificially derived polypeptides or polypeptide encoding nucleic acids that are not naturally occurring. Synthetic polypeptides and/or nucleic acids may be assembled de novo from basic subunits including, e.g., single amino acids, single nucleotides, etc., or may be derived from pre-existing polypeptides or polynucleotides, whether naturally or artificially derived, e.g., as through recombinant methods. Chimeric and engineered polypeptides or polypeptide encoding nucleic acids will generally be constructed by the combination, joining or fusing of two or more different polypeptides or polypeptide encoding nucleic acids or polypeptide domains or polypeptide domain encoding nucleic acids. Chimeric and engineered polypeptides or polypeptide encoding nucleic acids include where two or more polypeptide or nucleic acid “parts” that are joined are derived from different proteins (or nucleic acids that encode different proteins) as well as where the joined parts include different regions of the same protein (or nucleic acid encoding a protein) but the parts are joined in a way that does not occur naturally.

The term “recombinant” as used herein describes a nucleic acid molecule, e.g., a polynucleotide of genomic, cDNA, viral, semisynthetic, and/or synthetic origin, which, by virtue of its origin or manipulation, is not associated with all or a portion of the polynucleotide sequences with which it is associated in nature. The term recombinant as used with respect to a protein or polypeptide means a polypeptide produced by expression from a recombinant polynucleotide. The term recombinant as used with respect to a host cell or a virus means a host cell or virus into which a recombinant polynucleotide has been introduced. Recombinant is also used herein to refer to, with reference to material (e.g., a cell, a nucleic acid, a protein, or a vector) that the material has been modified by the introduction of a heterologous material (e.g., a cell, a nucleic acid, a protein, or a vector).

The term “operably linked” refers to a juxtaposition wherein the components so described are in a relationship permitting them to function in their intended manner. For instance, a promoter is operably linked to a coding sequence if the promoter affects its transcription or expression. Operably linked nucleic acid sequences may but need not necessarily be adjacent. For example, in some instances a coding sequence operably linked to a promoter may be adjacent to the promoter. In some instances, a coding sequence operably linked to a promoter may be separated by one or more intervening sequences, including coding and non-coding sequences. Also, in some instances, more than two sequences may be operably linked including but not limited to e.g., where two or more coding sequences are operably linked to a single promoter.

A “constitutive promoter” is an unregulated promoter that allows for continual transcription of its associated gene. Examples of constitutive promoters include CMV, EF1a (elongation factor 1 alpha), SV40 (simian vacuolating virus 40), PGK1 (phosphoglycerate kinase), Ubc (ubiquitin C), beta actin, CAG (containing CMV enhancer, chicken beta actin promoter, and rabbit beta-globin splice acceptor), TRE (tetracycline response element), and CaMKIIa (Ca²⁺/calmodulin-dependent protein kinase II).

An “inducible promoter” is a regulated promoter that allows for the transcription of its associated genes only in the presence of a specific stimulus. Examples of inducible promoters include tetracycline-regulated promoter, steroid-regulated promoter, metal-regulated promoter, and estrogen receptor-regulated promoter.

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The terms “polypeptide,” “peptide,” and “protein” used interchangeably herein, refer to a polymeric form of amino acids of any length, which can include genetically coded and non-genetically coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like.

Polypeptides may be “non-naturally occurring” in that the entire polypeptide is not found in any naturally occurring polypeptide. It will be understood that components of non-naturally occurring polypeptides may be naturally occurring, including but not limited to domains (such as functional domains) that may be included in some embodiments.

A “vector” or “expression vector” is a replicon, such as plasmid, phage, virus, or cosmid, to which another DNA segment, i.e. an “insert,” may be attached so as to bring about the replication of the attached segment in a cell.

The terms “domain” and “motif,” used interchangeably herein, refer to both structured domains having one or more functions and unstructured segments of a polypeptide that, although unstructured, retain one or more functions. For example, a structured domain may encompass but is not limited to a continuous or discontinuous plurality of amino acids, or portions thereof, in a folded polypeptide that comprise a three-dimensional structure which contributes to a function of the polypeptide. In other instances, a domain may include an unstructured segment of a polypeptide comprising a plurality of two or more amino acids, or portions thereof, that maintains a function of the polypeptide unfolded or disordered. Also encompassed within this definition are domains that may be disordered or unstructured but become structured or ordered upon association with a target or binding partner. Non-limiting examples of intrinsically unstructured domains and domains of intrinsically unstructured proteins are described, e.g., in Dyson & Wright. Nature Reviews Molecular Cell Biology 6:197-208.

The term “binding” refers to a direct association between two molecules, due to, for example, covalent, electrostatic, hydrophobic, and ionic and/or hydrogen-bond interactions, including interactions such as salt bridges and water bridges. Non-specific binding would refer to binding with an affinity of less than about 10⁷ M, e.g., binding with an affinity of 10⁶ M, 10⁵ M, 10⁴ M, etc.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease.

“Treatment,” as used herein, covers any treatment of a disease in a mammal, e.g., in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The terms “individual,” “subject,” “host,” and “patient” used interchangeably herein, refer to a mammal, including, but not limited to, murines (e.g., rats, mice), lagomorphs (e.g., rabbits), non-human primates, humans, canines, felines, ungulates (e.g., equines, bovines, ovines, porcines, caprines), etc.

A “therapeutically effective amount” or “efficacious amount” refers to the amount of an agent, or combined amounts of two agents, that, when administered to a mammal or other subject for treating a disease, is sufficient to effect such treatment for the disease. The “therapeutically effective amount” will vary depending on the agent(s), the disease and its severity and the age, weight, etc., of the subject to be treated.

The terms “chimeric antigen receptor” and “CAR,” used interchangeably herein, refer to artificial multi-module molecules capable of triggering or inhibiting the activation of an immune cell which generally but not exclusively comprise an extracellular domain (e.g., a ligand/antigen binding domain), a transmembrane domain and one or more intracellular signaling domains.

The term CAR is not limited specifically to CAR molecules but also includes CAR variants. CAR variants include split CARs wherein the extracellular portion (e.g., the ligand binding portion) and the intracellular portion (e.g., the intracellular signaling portion) of a CAR are present on two separate molecules. CAR variants also include ON-switch CARs which are conditionally activatable CARs, e.g., comprising a split CAR wherein conditional hetero dimerization of the two portions of the split CAR is pharmacologically controlled (e.g., as described in PCT publication no. WO 2014/127261 and US Patent Application No. 2015/0368342, the disclosures of which are incorporated herein by reference in their entirety). CAR variants also include bispecific CARs, which include a secondary CAR binding domain that can either amplify or inhibit the activity of a primary CAR. CAR variants also include inhibitory chimeric antigen receptors (iCARs) which may, e.g., be used as a component of a bispecific CAR system, where binding of a secondary CAR binding domain results in inhibition of primary CAR activation. CAR molecules and derivatives thereof (i.e., CAR variants) are described, e.g., in PCT Application No. US2014/016527; Fedorov et al. Sci Transl Med (2013); 5(215):215ra172; Glienke et al. Front Pharmacol (2015) 6:21; Kakarla & Gottschalk 52 Cancer J (2014) 20(2): 151-5; Riddell et al. Cancer J (2014) 20(2): 141-4; Pegram et al. Cancer J (2014) 20(2): 127-33; Cheadle et al. Immunol Rev (2014) 257(1):91-106; Barrett et al. Annu Rev Med (2014) 65:333-47; Sadelain et al. Cancer Discov (2013) 3(4):388-98; Cartellieri et al., J Biomed Biotechnol (2010) 956304; the disclosures of which are incorporated herein by reference in their entirety. Useful CARs also include the anti-CD19-4-1BB-CD3z CAR expressed by lentivirus loaded CTL019 (Tisagenlecleucel-T) CAR-T cells as commercialized by Novartis (Basel, Switzerland). The terms “chimeric antigen receptor” and “CAR” also include SUPRA CAR and PNE CAR (see, e.g., Cho et al Cell 2018 173: 1426-1438 and Rodgers et al, Proc. Acad. Sci. 2016 113: E459-468).

The terms “T cell receptor” and “TCR” are used interchangeably and will generally refer to a molecule found on the surface of T cells, or T lymphocytes, that is responsible for recognizing fragments of antigen as peptides bound to major histocompatibility complex (MHC) molecules. The TCR complex is a disulfide-linked membrane-anchored heterodimeric protein normally consisting of the highly variable alpha (a) and beta (b) chains expressed as part of a complex with CD3 chain molecules. Many native TCRs exist in heterodimeric ab or gd forms. The complete endogenous TCR complex in heterodimeric ab form includes eight chains, namely an alpha chain (referred to herein as TCRa or TCR alpha), beta chain (referred to herein as ‘H¾b or TCR beta), delta chain, gamma chain, two epsilon chains and two zeta chains. In some instance, a TCR is generally referred to by reference to only the TCRa and TCRb chains, however, as the assembled TCR complex may associate with endogenous delta, gamma, epsilon and/or zeta chains an ordinary skilled artisan will readily understand that reference to a TCR as present in a cell membrane may include reference to the fully or partially assembled TCR complex as appropriate.

Recombinant or engineered individual TCR chains and TCR complexes have been developed. References to the use of a TCR in a therapeutic context may refer to individual recombinant TCR chains. As such, engineered TCRs may include individual modified TCRa or modified TCRb chains as well as single chain TCRs that include modified and/or unmodified TCRa and TCRP chains that are joined into a single polypeptide by way of a linking polypeptide.

The terms “synthetic Notch receptor,” “synNotch,” and “synNotch receptor,” used interchangeably herein, refer to recombinant chimeric binding-triggered transcriptional switches that include at least: an extracellular binding domain, a portion of a Notch receptor that includes at least one proteolytic cleavage site, and an intracellular domain that provides a signaling function. SynNotch polypeptides, the components thereof and methods of employing the same, are described in U.S. Pat. Nos. 9,834,608 and 9,670,281, as well as, Toda et al., Science (2018) 361(6398): 156-16; Roybal & Lim, Annu Rev Immunol. (2017) 35:229-253; Lim & June Cell. (2017) 168(4):724-740; Roybal et al. Cell. (2016) 167(2):419-432.e16; Roybal et al. Cell. (2016) 164(4):770-9; and Morsut et al. Cell. (2016) 164(4):780-91; the disclosures of which are incorporated herein by reference in their entirety.

The terms “exogenous” and “external” are used interchangeably herein.

Before the present invention is further described, it is to be understood that this invention is not limited to embodiments described, as such may, of course, vary. It is also to be understood that the terminology used herein is for the purpose of describing certain embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a circuit” includes a plurality of such circuits and reference to “the nucleic acid” includes reference to one or more nucleic acids and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

Certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION OF THE INVENTION

The disclosure relates to molecular circuits, cells comprising such molecular circuits and methods of using the cells in controlling cellular behaviors, for example, in controlling cellular behavior in cellular therapies.

As noted above, provided herein are a variety of new cellular circuits, including feed forward circuits. Feed forward circuits can be implemented in a variety of different ways. In many implementations, a feed forward circuit contains a branched signal transduction pathway in which the first branch is engineered to inhibit the second branch, without inhibiting the first branch. For example, in some embodiments, activation of a signal transduction pathway may activate or induce the expression of a transcription factor which, in turn, generates a cellular response (e.g., differentiation or T cell activation). In this example, a construct could be added to the cell, where the construct contains a promoter that is also activated by the transcription factor (which construct “branches” the signal transduction pathway), operably linked to a coding sequence for an engineered regulatory protein that can inhibit the activity of a downstream protein, i.e., a protein that is downstream of the transcription factor and part of the cellular response. In these embodiments, the downstream protein may itself be modified in order to be selectively targeted by the engineered regulatory protein. For example, in the case of an immune cell such as a T cell, a construct containing an NFAT-dependent promoter operably linked to a coding sequence for an engineered regulatory protein could be introduced into a T cell, where the regulatory protein targets one or more of the proteins that are induced by NFAT (e.g., CDK4, cyclin A2, or any of the other proteins that are induced by NFAT). In another example, engineered cells often already contain engineered promoters (e.g., GAL4-responsive promoters) that drive the expression of a target protein. In this example, one could add another construct containing the GAL4-responsive promoter operably linked to an engineered regulatory protein that degrades the target protein. In this example, the engineered regulatory protein and the target protein may be designed to interact with one another.

As would be apparent from this disclosure, feed forward circuits can be implemented in a variety of different ways, e.g., to provide a “pulse” of the target protein or to act as a filter, etc, as described in the examples. For example, in some embodiments, expression of the engineered regulatory protein may be delayed relative to the target protein, thereby allowing a “pulse” of the target protein. Other configurations are possible. In any embodiment, the target protein may itself be a component of a signal transduction pathway and, a such, the circuit may be implemented in a way that provides a pulse of signaling, for example.

The engineered regulatory protein may inhibit the activity of the target protein by any suitable mechanism. For example, the target protein may contain a caged degron, in which case the engineered regulatory protein might be a “key” that opens the LOCKR, exposes the degron, and causes the target protein to be degraded by the proteosome degradation. In another example, the target protein may contain a degron and the engineered regulatory protein may be a protease that cleaves the target protein and exposes the degron, thereby causing the target protein to be degraded by the proteosome degradation. In another example, the engineered regulatory protein may bind to the target protein and inhibit its activity in a dominant negative manner, or the engineered regulatory protein may sequester the target protein to a site within the cell that it can't act, e.g., at the plasma membrane or the like. In another embodiment, the engineered regulatory protein may carry a degron and binding of the engineered regulatory protein to the target protein targets the target protein for degradation in trans.

As noted above, such a circuit may comprise a first nucleic acid comprising a first nucleotide sequence encoding an inactivating protein that is activated by a first exogenous stimulus, and a second nucleic acid comprising a second nucleotide sequence encoding a target protein that is activated by the first exogenous stimulus, where the target protein has a regulatory motif and the activated inactivating protein inactivates the target protein via the regulatory motif. In some embodiments, activation of the inactivating protein can be delayed relative to the activation of the target protein. As described below, the delay may be implemented in a variety of different ways and, in some embodiments, the delay can be modulated to provide a particular effect. In some embodiments, the delay may be at least 5 minutes, at least 10 minutes, at least 15 minutes, at least 30 minutes, or least 1 hour, which delay allows signaling to occur for a period of time before being shut down.

In some embodiments, the cell may comprise a molecular circuit comprising: a nucleic acid encoding a target protein that comprises a regulatory motif that is not native to the target protein (e.g., not part of the protein in its wild type form, if the protein is otherwise naturally occurring); a nucleic acid encoding an inactivating protein, wherein the inactivating protein binds to the regulatory motif and inactivates the target protein; and an actuating protein that, in response to a first external stimulus, independently activates expression the target protein of (a) and the inactivating protein of (b). In this description, the term “independently” means that activation of one is not dependent on activation of the other. This can be done via a branched signal transduction pathway. One of these components is not upstream from the other. In some embodiments, the actuating protein may independently activate transcription of the nucleic acid of (a) and the nucleic acid of (b) in response to the exogenous stimulus. In these embodiments, the actuating protein may be a transcription factor (e.g., a transcription factor that is activated by compound such as an estrogen), although it could be another type of protein. In some embodiments, the exogenous stimulus may bind to the actuating protein or to a protein that is upstream of the actuating protein (in the same signal transduction pathway as the actuating protein), wherein binding directly or indirectly activates the actuating protein. In some embodiments, the exogenous stimulus may induce expression of the actuating protein. In some embodiments, the exogenous stimulus may bind to a transmembrane protein on the outside of the cell. In these latter embodiments, binding may initiates a signal transduction cascaded that results in activation of the actuating protein inside the cell. The exogenous stimulus can be an added compound (a drug), a compound produced by a neighboring cell, or a binding event that occurs outside of the cell that is transduced to the inside of a cell (e.g., to a transmembrane receptor).

As noted above, in some embodiments, activation of expression of the inactivating protein by the actuating protein is delayed relative to activation of expression of the target protein by the actuating protein. This delay may be implemented in a variety of different ways. For example, in some embodiments, the target protein is directly activated by the actuating protein whereas the target protein is indirectly activated by the actuating protein (e.g., via a transcriptional cascade, for example).

In some embodiments, the cell may further comprise (d) a controller protein that controls the interaction between the inactivating protein of (b) and the target protein of (a). This may be done by binding to or inactivating the inactivating protein or by blocking the interaction between the inactivating protein and the target protein. In these embodiments, the expression and/or activity of the controller protein may be modulated by a second exogenous stimulus, e.g., a second compound that may be supplied exogenously.

The target protein may be any protein. However, in some embodiments, the target protein may be an engineered cell surface receptor (e.g., a CAR), an intracellular kinase or engineered transcription factor. In some cases, the target protein may a member of a signal transduction cascade that, when activated, induces in a change of expression of proteins that are endogenous to the cell; (e.g., T cell activation, cytokine production, differentiation, cell division, apoptosis, etc.). In these embodiments, the inactivating protein may be incapable of modulating any endogenous signal transductions in the cell, except by inactivating the target protein.

The inactivating protein may inactivate the target protein in a variety of different ways. For example, in some embodiments, the inactivating protein may induce degradation of the target protein. In these embodiments, the target protein may comprise a caged degron and the inactivating protein comprises a molecular key that exposes the degron, thereby causing degradation of the target protein. In other embodiments, the inactivating protein may itself comprise a degron and binding of the inactivating protein to the targeting protein causes degradation of the target protein in trans. Alternatively, the target protein may contain an internal degron that is next to a protease cleavage site. In these embodiments, the inactivating protein may be a protease that cleaves at the protease cleavage site and activates the degron, thereby causing degradation of the target protein. In other embodiments, the inactivating protein may comprise a sub-cellular targeting domain, and binding of inactivating protein to the target protein sequesters the target protein (e.g., to the plasma membrane, for example). Alternatively, the inactivating protein may inhibit the target protein in a dominant negative manner.

For example, dynamic regulation of CAR activation in T cells has been identified as a potential means to not only reduce toxicity, but also to enhance therapeutic efficacy by mitigating exhaustion. Certain molecular circuits disclosed herein can be used for such regulation of CAR-T cell activation.

Also, the activation of CAR-T cells may have to be limited to only certain densities of target antigens. Often, cancerous cells have higher densities of certain antigens, such as Her2. Current receptor designs cannot distinguish between healthy cells and cancerous cells based on cancer antigen expression. Certain molecular circuits disclosed herein can be used in CAR-T cells to distinguish between healthy cells and cancerous cells based on the level of cancer antigen expression.

In general, any system with toxic intermediates can potentially benefit from pulsing behavior. Gene editing using CRISPR-Cas9 is known to cause off target or unintended mutations in cells that could lead to dangerous outcomes like activating an oncogene. Degradation of Cas9 has been shown to reduce these off-target mutations by decreasing the half-life of Cas9. Also, Cas9 can be knocked off the DNA after Cas9 has made a cut, which can reduce toxicity of Cas9 by making it easier for DNA repair machinery to repair the broken DNA. Certain embodiments of the invention can be used to build molecular circuits that produce an initial pulse of Cas9 that is then degraded to further reduce Cas9 toxicity while maintaining efficiency by not decreasing the peak expression.

Thus, stem cells and other cell types engineered according to this disclosure can be used in regenerative medicine. Circuits such as a concentration filter could be used to pattern mammalian cells to facilitate synthetic organ production or controlled differentiation of stem cells.

For systems that are sensitive to noise in regulator, antigen, or DNA concentrations, the molecular circuits disclosed herein can be inserted to buffer against that noise by either delaying an unwanted response or detecting a fold change instead of absolute concentrations. This could be used to engineer cell sensors to adapt to changing environments such as cell therapies to tackle tumor heterogeneity. Additionally, fold change detection could help networks adapt to unpredictable DNA concentration. Some genetic engineering methods can lead to multiple insertions when modifying DNA. The molecular circuits disclosed herein could reduce the noise caused by these methods.

Certain embodiments of this disclosure use programmed signal processing units, for example, signal processing units using the caged-degron switch technology degronLOCKR. LOCKR switch technology utilizes a peptide key that activates in degronLOCKR containing proteins a switch containing a signaling peptide. The signaling peptide can induce degradation of the protein or translocation of the protein to another cellular compartment.

The designer nature of degronLOCKR makes it a powerful tool for controlling the activity of nearly any protein, for example, via degradation or translocation of the protein. In certain embodiments of the invention, degronLOCKR is used to program synthetic feedback and feedforward on arbitrary nodes in a circuit to generate different types of cellular behavior. Furthermore, designer decoys can be introduced for the key to sequester and deactivate the activity of the key.

A unique characteristic of the degronLOCKR system is the ability to mix feedback and feedforward (including control through the decoy) in a combinatorial fashion in a single circuit. Orthogonal LOCKR systems caging the same, or different, signaling peptides could be used in the circuits to create even more diverse cellular behaviors. In certain embodiments, a library of circuits implementing pulse generation and band-pass concentration filters and utilizing the degronLOCKR system are provided.

Certain aspects of the degronLOCKR system are described in PCT Publications WO2020146254, WO2020146260, and WO2020154087. These publications are incorporated by reference in their entireties, particularly, the Sequence Listings in these publications.

Alternatively, the inactivating protein may comprise: (a) a target-binding domain (e.g., a scFv, nanobody, or dimerization domain such as a synthetic leucine zipper domain) that binds to the target protein (where the regulatory motif may contain a binding partner for the target-binding domain, e.g., a complementary synthetic leucine zipper domain); and (b) a ubiquitination-recruiting domain that is heterologous to the target-binding domain (e.g., a degron or an E3 ligase-recruiting domain that binds directly or indirectly (via an adapter protein) to an E3 ligase), where binding of the fusion protein to a target protein via the target-binding domain induces degradation of the target protein via the ubiquitination-mediated degradation. Examples of such fusion proteins (which may be lysine-free in some instances) that could potentially be employed are described in PCT/US2021/47391 filed Aug. 24, 2021, and others. For example, in some embodiments, the target binding protein may contain a first member of a dimerization pair (e.g., a first synthetic leucine zipper) and the inactivating protein may contain a second member of a dimerization pair (e.g., a first synthetic leucine zipper) where the first and second members dimerize, as well as a C-terminal degron sequence (e.g., Arg-Arg-Arg-Gly; also referred to as the “Bonger” motif). This molecule may be lysine-free and targets other proteins for degradation in trans (i.e., by binding to them). In these embodiments, the regulatory motif of the target protein (which may be added onto the target protein) may be referred to as an “inactivating protein recruiting domain” since it binds to the inactivating protein, which, in turn, causes its own degradation. Examples of dimerization pairs include synZips, coiled-coil pairs and helix-turn-helix (or “designed heterodimer”) pairs, although many others are known.

### Molecular Circuits

Molecular circuits of the present disclosure may, in some instances and in whole or in part, be encoded by nucleic acid sequences. Such circuits may, in some instances, be present and/or configured in expression vectors and/or expression cassettes. The subject nucleic acids of the present circuits may, in some instances, be contained within a vector, including e.g., viral and non-viral vectors. Such circuits may, in some instances, be present in cells, such as immune cells, stem cells, etc., or may be introduced into cells by various means, including e.g., a viral vector. Cells may, in some instances, be genetically modified to contain and/or encode a subject circuit, where such modification may be effectively permanent (e.g., integrated) or transient as desired. Circuits of the present disclosure, the components of which are modular, may include a target protein that includes a caged degron.

As used herein, the term “target protein” generally refers to a protein of a signaling pathway, including natural and synthetic signaling pathways. Any convenient and appropriate target protein of any convenient signaling pathway may be employed. Generally, target proteins include proteins that may be activated by an input of the signaling pathway with which the target protein is associated. A signaling pathway may generate an output that is dependent upon, or at least influenced by, the function of the target protein. Such outputs may be a direct or indirect result of the response of the target protein to the input. Useful target proteins include members from any convenient and appropriate point a signaling pathway, including input-receiving members, intermediate members, and output-producing members.

By “input-receiving members,” as used herein, is generally meant the initial component of a signaling pathway that receives an input to initiate signaling along the pathway. Examples of input-receiving members include but are not limited to e.g., extracellular receptors (e.g., G protein-coupled receptors, protein kinases, integrins, toll-like receptors, ligand-gated ion channels, and the like) and intracellular receptors (e.g., nuclear receptors, cytoplasmic receptors, etc.). In some instances, an input-receiving member may be a protein that directly binds an input of a signaling pathway, such as a ligand input of a signaling pathway. In some instances, a target protein that includes a caged degron in a circuit of the present disclosure may be an input-receiving member. In some instances, a target protein that includes a caged degron in a circuit of the present disclosure may not be an input-receiving member, e.g., it may be an intermediate member or an output-producing member.

By “intermediate member,” as used herein, is generally meant a component of a signaling pathway that is required for, or at least involved in, signal transduction but does not directly receive the initial input or directly produce or cause the final output of the signaling pathway. Examples of intermediate members of a signaling pathway include but are not limited to e.g., enzymes, binding partners, protein complex subunits, scaffold proteins, transport proteins, co-activators, co-repressors, and the like. In some instances, a target protein that includes a caged degron in a circuit of the present disclosure may be an intermediate member. In some instances, a target protein that includes a caged degron in a circuit of the present disclosure may not be an intermediate member, e.g., it may be an input-receiving member or an output-producing member.

By “output-producing member,” as used herein, is generally meant a component of a signaling pathway that directly produces an output of the signaling pathway or otherwise causes the output of the signaling pathway to occur. Examples of output-producing members of a signaling pathway include but are not limited to e.g., DNA binding proteins, such as e.g., transcription factors, enzymes, and the like. In some instances, a target protein that includes a caged degron in a circuit of the present disclosure may be an output-producing member. In some instances, a target protein that includes a caged degron in a circuit of the present disclosure may not be an output-producing member, e.g., it may be an input-receiving member or an intermediate member.

A target protein employed in a circuit of the present disclosure may, when activated, drive an output of the signaling pathway. As such, degradation of the target protein, may negatively regulate the output of the signaling pathway. A target protein employed in a circuit of the present disclosure may, when activated, inhibit an output of the signaling pathway. As such, degradation of the target protein, may positively regulate the output of the signaling pathway.

Target proteins that may be employed in the circuits of the present disclosure include target proteins that are endogenous components of the signaling pathway as well as heterologous or synthetic components of the signaling pathway. Such endogenous, heterologous and/or synthetic components of signaling pathways may be modified to include a caged degron, described in more detail below, for use in a circuit of the present disclosure.

By “endogenous component of the signaling pathway” is generally meant a component of the signaling pathway as it occurs naturally in a cell.

By “heterologous component of the signaling pathway” is generally meant a component that functions in the signaling pathway but is derived from a cell or signaling pathway other than that in which it is employed in the subject circuit. Heterologous components may be derived from a signaling pathway separate from the signaling pathway of the subject circuit. Heterologous components may be derived from a different type of cell and/or a different organism from the cell and/or organism of the signaling pathway modulated in the subject circuit. For example, in some instances, a component of a signaling pathway from a first organism (e.g., mouse) may be employed in a corresponding signaling pathway of a second organism (e.g., human).

By “synthetic component of the signaling pathway” is generally meant a component that functions in the signaling pathway but is non-naturally derived. Non-naturally derived components may include recombinant components, including e.g., analogs, mimetics, fusions, mutants, truncated versions, fragments, and the like. Non-limiting examples of synthetic components of signaling pathways including synthetic receptors, synthetic enzymes, synthetic co-activators, synthetic co-repressors, synthetic binding partners, synthetic scaffold proteins, synthetic transcription factors, and the like.

Circuits of the present disclosure may employ one or more regulatory sequences, the control of which may be dependent upon a component of the signaling pathway with which the target protein is associated. For example, in some instances, a circuit of the present disclosure may include a regulatory sequence responsive to an output of the signaling pathway. Regulatory sequences may be operably linked to one or more nucleic acid sequences encoding one or more components of the subject circuit. For example, a regulatory sequence may be operably linked to a nucleic acid sequence encoding an inactivating protein, such as a key polypeptide.

In some instances, a circuit may include a promoter sequence operably linked to a nucleic acid sequence encoding the target protein. Regulatory sequences operably linked to a sequence encoding the target protein of the subject circuits may vary and may include endogenous and heterologous regulatory sequences, including but not limited to e.g., native promoters, native enhancers, heterologous promoters, heterologous enhancers, synthetic regulatory sequences, and the like. Regulatory sequences operably linked to a sequence encoding the target protein may be constitutive or inducible as desired. In some instances, a regulatory sequence operably linked to the nucleic acid sequence encoding a target protein is a native promoter of the target protein.

In some instances, a regulatory sequence may include one or more binding sites (e.g., 1 or more, 2 or more, 2 to 10, 3 to 10, 4 to 10, 5 to 10, 2 to 6, 3 to 6, 4 to 6, 1, 2, 3, 4, 5, 6, 7, 8, 9, 10, etc.) for one or more transcription factors e.g., where the transcription factor is an endogenous, heterologous, or synthetic transcription factor that functions in the signaling pathway.

Regulatory sequences of circuits of the present disclosure may be controlled by, or otherwise responsive to, an output of a signaling pathway. For example, in some instances, an output of a signaling pathway, which the subject circuit is configured to influence, may induce expression of a coding sequence through a regulatory sequence operably linked to the coding sequence. By connecting the regulation of a sequence encoding a component of a circuit of the present disclosure to an output of the signaling pathway, circuits of the present disclosure may provide feedback that is response to the output.

Useful signaling pathway outputs employed in circuits of the present disclosure may vary and may include essentially any output that may be configured to directly or indirectly influence expression through a regulatory sequence. Non-limiting examples of useful signaling pathway outputs include but are not limited to e.g., activity (e.g., activation, repression, etc.) of a transcription factor, expression of a transcription factor, translocation of a transcription factor, activity (e.g., activation, repression, etc.) of an enzyme, expression of an enzyme, production of a signaling molecule, secretion of a signaling molecule, cellular activation (including e.g., activation of native cellular programs, such as but not limited to e.g., immune activation, immune suppression, proliferation, etc.), and the like.

Signaling pathways may be modulated (e.g., activated, repressed, etc.) by one or more inputs. Inputs of signaling pathways may vary and may include endogenous (e.g., native) inputs of signaling pathways and heterologous (e.g., engineered or synthetic) signaling pathway inputs. As signaling pathways, and signaling pathway outputs, may be native or synthetic, signaling pathway inputs may similarly be native or synthetic.

Native signaling pathways may be controlled by a native or natural receptor of the pathway. Non-limiting examples of native signaling pathways include but are not limited to e.g., the AKT signaling pathway, the Akt/PKB signaling pathway, the AMPK signaling pathway, the apoptosis signaling pathway, the BMP signaling pathway, the cAMP-dependent pathway, the estrogen signaling pathway, the hedgehog signaling pathway, the hippo signaling pathway, an immune activation pathway, an immune suppression pathway, an immune cell differentiation pathway, an insulin signal transduction pathway, the JAK-STAT signaling pathway, the MAPK/ERK signaling pathway, the mTOR signaling pathway, the NF-KB signaling pathway, the nodal signaling pathway, the notch signaling pathway, the p53 signaling pathway, the PI3K signaling pathway, the TGF beta signaling pathway, the TFR signaling pathway, the TNF signaling pathway, the VEGF signaling pathway, the Wnt signaling pathway, and the like.

Non-limiting examples of synthetic signaling pathways include, but are not limited to, those pathways controlled by a synthetic or engineered receptor, such as but not limited to a CAR, an engineered TCR, a synNotch, etc. Suitable non-limiting examples of synthetic signaling pathways that may be modulated using a circuit of the present disclosure also include biosynthesis and/or bioproduction pathways. Biosynthesis and/or bioproduction pathways may be natural or synthetic and may be employed in cells and/or organisms where the pathway is endogenous or heterologous.

In some instances, a pathway modulated using a circuit of the present disclosure may include an immune modulation pathway, such as e.g., an immune activation pathway or an immune suppression pathway. Such immune modulation pathways may be natural or synthetic and may be endogenous to the cell in which the circuit is employed or heterologous to the cell in which the circuit is employed.

Nucleic Acids

The present disclosure also provides nucleic acids encoding molecular circuits disclosed herein. The subject nucleic acids may include, e.g., a sequence encoding a target protein, an inactivating protein, or a controller protein. Such nucleic acids may be configured such that one or more of the sequences are operably linked to a regulatory sequence. For example, a nucleic acid may be configured such that the sequence encoding a target protein is operably linked to a regulatory sequence responsive to an output of the signaling pathway. Provided are nucleic acids encoding the circuits described herein. Encompassed are isolated nucleic acids encoding the subject circuits as well as various configurations containing such nucleic acids, such as vectors, e.g., expression cassettes, recombinant expression vectors, viral vectors, and the like.

Recombinant expression vectors of the present disclosure include those comprising one or more of the described nucleic acids. A nucleic acid comprising a nucleotide sequence encoding all or a portion of the components of a circuit of the present disclosure will in some embodiments be DNA, including, e.g., a recombinant expression vector. A nucleic acid comprising a nucleotide sequence encoding all or a portion of the components of a circuit of the present disclosure will in some embodiments be RNA, e.g., in vitro synthesized RNA.

In some instances, the subject circuits may make use of an encoding nucleic acid (e.g., a nucleic acid encoding a target protein) that is operably linked to a regulatory sequence such as a transcriptional control element (e.g., a promoter; an enhancer; etc.). In some cases, the transcriptional control element is inducible. In some cases, the transcriptional control element is constitutive. In some cases, the promoters are functional in eukaryotic cells. In some cases, the promoters are functional in prokaryotic cells. In some cases, the promoters are cell type-specific promoters. In some cases, the promoters are tissue-specific promoters.

Depending on the host/vector system utilized, any of a number of suitable transcription and translation control elements, including constitutive and inducible promoters, transcription enhancer elements, transcription terminators, etc. may be used in the expression vector (see e.g., Bitter et al. (1987) Methods in Enzymology, 153:516-544).

A promoter can be a constitutively active promoter (i.e., a promoter that is constitutively in an active/“ON” state), it may be an inducible promoter (i.e., a promoter whose state, active/“ON” or inactive/“OFF,” is controlled by an exogenous stimulus, e.g., the presence of a particular temperature, compound, or protein), it may be a spatially restricted promoter (i.e., transcriptional control element, enhancer, tissue specific promoter, cell type specific promoter, etc.), and it may be a temporally restricted promoter (i.e., the promoter is in the “ON” state or “OFF” state during specific stages of embryonic development or during specific stages of a biological process, e.g., the cell cycle, the hair follicle cycle in mammals, circadian cycles in mammals, etc.).

Suitable promoter and enhancer elements are known in the art. For expression in a bacterial cell, suitable promoters include, but are not limited to, lad, lacZ, T3, T7, gpt, lambda P and trc. For expression in a eukaryotic cell, suitable promoters include, but are not limited to, yeast promoters (e.g., promoters of yeast mating pathway genes, yeast galactose-inducible promoters, etc.), light and/or heavy chain immunoglobulin gene promoter and enhancer elements; cytomegalovirus immediate early promoter; herpes simplex virus thymidine kinase promoter; early and late SV40 promoters; promoters present in long terminal repeats from a retrovirus; mouse metallothionein-I promoter; and various art-known tissue specific promoters.

In some instances, transcriptional control elements of varied strength may be employed. For example, promoters, e.g., constitutive or inducible promoters, of varied strength, such as e.g., weak, intermediate, and strong promoters, such as but not limited to e.g., constitutive promoters pREV1, pRNR2, pRET2, etc. may be employed. In some instances, the strength of a promoter may be modulated, e.g., made weaker or made stronger, by decreasing or increasing, respectively, the number of binding sites (e.g., DBD binding sites) within the promoter.

In some instances, a transcriptional control element of a herein described nucleic acid may include a cis-acting regulatory sequence. Any suitable cis-acting regulatory sequence may find use in the herein described nucleic acids. For example, in some instances a cis-acting regulatory sequence may be or include an upstream activating sequence or upstream activation sequence (UAS). In some instances, a UAS of a herein described nucleic acid may be a Gal4 responsive UAS.

In some instances, transcriptional control of a circuit of the present disclosure may include the use of one or more regulatory sequences responsive to a synthetic transcription factor. Synthetic transcription factors, and regulatory sequences responsive thereto, will vary and may include but are not limited to e.g., estradiol ligand binding domain (LBD) based synthetic transcription factors, progesterone LBD based synthetic transcription factors, zinc-finger based synthetic transcription factors, and the like. Synthetic transcription factors may by chimeric and may include various domains, e.g., a DNA binding domain (DBD), activation domain, zinc-finger domain(s), and the like. Useful domains, e.g., LBDs, DBDs, activation domains, etc., will vary and may include but are not limited to e.g., the Gal4p DBD, the Zif268 transcription factor DBD, viral activation domains (e.g., VP16, VP64, etc.), Msn2p activation domains, and the like. Non-limiting examples of useful synthetic transcription factors include but are not limited to e.g., GEM (Gal4 DNA binding domain-Estradiol hormone binding domain-Msn2 activation domain), Z3PM (Z3 zinc finger-Progesterone hormone binding domain-Msn2 activation domain), and the like.

Correspondingly, useful regulatory sequences will vary and may include promoters responsive to synthetic transcription factors, including but not limited to e.g., pZ promoters, pZ3 promoters, pGAL1 promoters, and the like. Examples of suitable promoters and synthetic transcription factors include, but are not limited to e.g., those described herein, those described in Aranda-Diaz et al. ACS Synth Biol. (2017) 6(3): 545-554; the disclosure of which is incorporated herein by reference in its entirety, and the like.

Suitable promoters may, in some instances, include suitable reversible promoters. Reversible promoters may be isolated and derived from many organisms, e.g., eukaryotes and prokaryotes. Modification of reversible promoters derived from a first organism for use in a second organism, e.g., a first prokaryote and a second a eukaryote, a first eukaryote and a second a prokaryote, etc., is well known in the art. Such reversible promoters, and systems based on such reversible promoters but also comprising additional control proteins, include, but are not limited to, alcohol regulated promoters (e.g., alcohol dehydrogenase I (ale A) gene promoter, promoters responsive to alcohol transactivator proteins (AlcR), etc.), tetracycline regulated promoters, (e.g., promoter systems including TetActivators, TetON, TetOFF, etc.), steroid regulated promoters (e.g., rat glucocorticoid receptor promoter systems, human estrogen receptor promoter systems, retinoid promoter systems, thyroid promoter systems, eedysone promoter systems, mifepristone promoter systems, etc.), metal regulated promoters (e.g., metallothionein promoter systems, etc.), pathogenesis-related regulated promoters (e.g., salicylic acid regulated promoters, ethylene regulated promoters, benzothiadiazole regulated promoters, etc.), temperature regulated promoters (e.g., heat shock inducible promoters (e.g., HSP-70, HSP-90, soybean heat shock promoter, etc.), light regulated promoters, synthetic inducible promoters, and the like.

Inducible promoters suitable for use include any inducible promoter described herein or known to one of ordinary skill in the art. Examples of inducible promoters include, without limitation, chemically/biochemically-regulated and physically-regulated promoters such as alcohol-regulated promoters, tetracycline-regulated promoters (e.g., anhydrotetracycline (aTc)-responsive promoters and other tetracycline-responsive promoter systems, which include a tetracycline repressor protein (tetR), a tetracycline operator sequence (tetO) and a tetracycline transactivator fusion protein (tTA)), steroid-regulated promoters (e.g., promoters based on the rat glucocorticoid receptor, human estrogen receptor, moth eedysone receptors, and promoters from the steroid/retinoid/thyroid receptor superfamily), metal-regulated promoters (e.g., promoters derived from metallothionein (proteins that bind and sequester metal ions) genes from yeast, mouse and human), pathogenesis-regulated promoters (e.g., induced by salicylic acid, ethylene or benzothiadiazole (BTH)), temperature/heat-inducible promoters (e.g., heat shock promoters), and light-regulated promoters (e.g., light responsive promoters from plant cells).

In some instances, a useful promoter may be an immune cell promoter. For example, in embodiments where components of a circuit are expressed in an immune cell, an immune cell promoter may be employed. Suitable immune cell promoters include but are not limited to e.g., CD8 cell-specific promoters, CD4 cell-specific promoters, neutrophil-specific promoters, and NK-specific promoters. For example, a CD4 gene promoter can be used; see, e.g., Salmon et al. (1993) Proc. Natl. Acad. Sci. USA 90: 7739; and Marodon et al. (2003) Blood 101:3416. As another example, a CD8 gene promoter can be used. NK cell-specific expression can be achieved by use of an Ncrl (p46) promoter; see, e.g., Eckelhart et al. (2011) Blood 117:1565.

In some instances, an immune cell specific promoter of a nucleic acid of the present disclosure may be a promoter of a B29 gene promoter, a CD 14 gene promoter, a CD43 gene promoter, a CD45 gene promoter, a CD68 gene promoter, a IFN-b gene promoter, a WASP gene promoter, a T-cell receptor b-chain gene promoter, a V9 g (TRGV9) gene promoter, a V2 d (TRDV2) gene promoter, and the like.

In some cases, a nucleic acid comprising a nucleotide sequence encoding a circuit of the present disclosure, or one or more components thereof, is a recombinant expression vector or is included in a recombinant expression vector. In some embodiments, the recombinant expression vector is a viral construct, e.g., a recombinant adeno-associated virus (AAV) construct, a recombinant adenoviral construct, a recombinant lentiviral construct, a recombinant retroviral construct, etc. In some cases, a nucleic acid comprising a nucleotide sequence encoding a circuit of the present disclosure, or one or more components thereof, is a recombinant lentivirus vector. In some cases, a nucleic acid comprising a nucleotide sequence encoding a circuit of the present disclosure, or one or more components thereof, is a recombinant AAV vector.

Suitable expression vectors include, but are not limited to, viral vectors (e.g. viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., Hum Gene Ther 5:1088 1097, 1999; WO 94/12649, WO 93/03769; WO 93/19191; WO 94/28938; WO 95/11984 and WO 95/00655); adeno-associated virus (see, e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et ah, J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like. In some cases, the vector is a lentivirus vector. Also suitable are transposon-mediated vectors, such as piggyback and sleeping beauty vectors.

In some instances, nucleic acids of the present disclosure may have a single sequence encoding two or more polypeptides where expression of the two or more polypeptides is made possible by the presence of a sequence element between the individual coding regions that facilitates separate expression of the individual polypeptides. Such sequence elements may be referred to herein as bicistronic-facilitating sequences, where the presence of a bicistronic-facilitating sequence between two coding regions makes possible the expression of a separate polypeptide from each coding region present in a single nucleic acid sequence. In some instances, a nucleic acid may contain two coding regions encoding two polypeptides present in a single nucleic acid with a bicistronic-facilitating sequence between the coding regions. Any suitable method for separate expression of multiple individual polypeptides from a single nucleic acid sequence may be employed and, similarly, any suitable method of bicistronic expression may be employed.

In some instances, a bicistronic-facilitating sequence may allow for the expression of two polypeptides from a single nucleic acid sequence that are temporarily joined by a cleavable linking polypeptide. In such instances, a bicistronic-facilitating sequence may include one or more encoded peptide cleavage sites. Suitable peptide cleavage sites include those of self-cleaving peptides as well as those cleaved by a separate enzyme. In some instances, a peptide cleavage site of a bicistronic-facilitating sequence may include a furin cleavage site (i.e., the bicistronic-facilitating sequence may encode a furin cleavage site).

In some instances, the bicistronic-facilitating sequence may encode a self-cleaving peptide sequence. Useful self-cleaving peptide sequences include but are not limited to e.g., peptide 2A sequences, including but not limited to e.g., the T2A sequence.

In some instances, a bicistronic-facilitating sequence may include one or more spacer encoding sequences. Spacer encoding sequences generally encode an amino acid spacer, also referred to in some instances as a peptide tag. Useful spacer encoding sequences include but are not limited to e.g., V5 peptide encoding sequences, including those sequences encoding a V5 peptide tag.

Multi- or bicistronic expression of multiple coding sequences from a single nucleic acid sequence may make use of but is not limited to those methods employing furin cleavage, T2A, and V5 peptide tag sequences. For example, in some instances, an internal ribosome entry site (IRES) based system may be employed. Any suitable method of bicistronic expression may be employed including but not limited to e.g., those described in Yang et al. (2008) Gene Therapy. 15(21): 1411-1423; Martin et al. (2006) BMC Biotechnology. 6:4; the disclosures of which are incorporated herein by reference in their entirety.

Cells

The present disclosure also provides cells containing nucleic acids encoding molecular circuits disclosed herein. Cells modified to include one or more nucleic acids encoding one or more molecular circuits and/or one or more components thereof may be referred to herein as having been genetically modified, where such modification may be stable or transient as desired. Useful cells may include prokaryotic and eukaryotic cells, including but not limited to e.g., bacterial cells, plant cells, animal cells, yeast cells, mammalian cells, rodent cells, non-human primate cells, human cells, and the like.

Suitable cells include stem cells, progenitor cells, as well as partially and fully differentiated cells. Suitable cells include, neurons, liver cells; kidney cells; immune cells; cardiac cells; skeletal muscle cells; smooth muscle cells; lung cells; and the like.

Suitable cells include a stem cell (e.g. an embryonic stem (ES) cell, an induced pluripotent stem (iPS) cell; a germ cell (e.g., an oocyte, a sperm, an oogonia, a spermatogonia, etc.); a somatic cell, e.g. a fibroblast, an oligodendrocyte, a glial cell, a hematopoietic cell, a neuron, a muscle cell, a bone cell, a hepatocyte, a pancreatic cell, etc.

Suitable cells include human embryonic stem cells, fetal cardiomyocytes, myofibroblasts, mesenchymal stem cells, autotransplated expanded cardiomyocytes, adipocytes, totipotent cells, pluripotent cells, blood stem cells, myoblasts, adult stem cells, bone marrow cells, mesenchymal cells, embryonic stem cells, parenchymal cells, epithelial cells, endothelial cells, mesothelial cells, fibroblasts, osteoblasts, chondrocytes, exogenous cells, endogenous cells, stem cells, hematopoietic stem cells, bone-marrow derived progenitor cells, myocardial cells, skeletal cells, fetal cells, undifferentiated cells, multi-potent progenitor cells, unipotent progenitor cells, monocytes, cardiac myoblasts, skeletal myoblasts, macrophages, capillary endothelial cells, xenogenic cells, allogenic cells, and post-natal stem cells.

In some cases, the cell is a stem cell. In some cases, the cell is an induced pluripotent stem cell. In some cases, the cell is a mesenchymal stem cell. In some cases, the cell is a hematopoietic stem cell. In some cases, the cell is an adult stem cell.

Suitable cells include bronchioalveolar stem cells (BASCs), bulge epithelial stem cells (bESCs), corneal epithelial stem cells (CESCs), cardiac stem cells (CSCs), epidermal neural crest stem cells (eNCSCs), embryonic stem cells (ESCs), endothelial progenitor cells (EPCs), hepatic oval cells (HOCs), hematopoetic stem cells (HSCs), keratinocyte stem cells (KSCs), mesenchymal stem cells (MSCs), neuronal stem cells (NSCs), pancreatic stem cells (PSCs), retinal stem cells (RSCs), and skin-derived precursors (SKPs).

In some instances, a cell is an immune cell. Suitable mammalian immune cells include primary cells and immortalized cell lines. Suitable mammalian cell lines include human cell lines, non-human primate cell lines, rodent (e.g., mouse, rat) cell lines, and the like. In some instances, the cell is not an immortalized cell line, but is instead a cell (e.g., a primary cell) obtained from an individual. For example, in some cases, the cell is an immune cell, immune cell progenitor or immune stem cell obtained from an individual. As an example, the cell is a lymphoid cell, e.g., a lymphocyte, or progenitor thereof, obtained from an individual. As another example, the cell is a cytotoxic cell, or progenitor thereof, obtained from an individual. As another example, the cell is a stem cell or progenitor cell obtained from an individual.

As used herein, the term “immune cells” generally includes white blood cells (leukocytes) which are derived from hematopoietic stem cells (HSC) produced in the bone marrow. “Immune cells” includes, e.g., lymphoid cells, i.e., lymphocytes (T cells, B cells, natural killer (NK) cells), and myeloid-derived cells (neutrophil, eosinophil, basophil, monocyte, macrophage, dendritic cells). “T cell” includes all types of immune cells expressing CD3 including T-helper cells (CD4+ cells), cytotoxic T-cells (CD8+ cells), T-regulatory cells (Treg) and gamma-delta T cells. A “cytotoxic cell” includes CD8+ T cells, natural-killer (NK) cells, and neutrophils, which cells are capable of mediating cytotoxicity responses. “B cell” includes mature and immature cells of the B cell lineage including e.g., cells that express CD19 such as Pre B cells, Immature B cells, Mature B cells, Memory B cells and plasmablasts. Immune cells also include B cell progenitors such as Pro B cells and B cell lineage derivatives such as plasma cells.

Cells encoding a molecular circuit of the present disclosure may be generated by any convenient method. Nucleic acids encoding one or more components of a molecular circuit may be stably or transiently introduced into the subject immune cell, including where the subject nucleic acids are present only temporarily, maintained extrachromosomally, or integrated into the host genome. Introduction of the subject nucleic acids and/or genetic modification of the subject immune cell can be carried out in vivo, in vitro, or ex vivo.

In some cases, the introduction of the subject nucleic acids and/or genetic modification is carried out ex vivo. For example, an immune cell, a stem cell, etc., is obtained from an individual; and the cell obtained from the individual is modified to express components of a circuit of the present disclosure. The modified cell can thus be modified with one or more signaling pathways of choice, as defined by the one or more molecular circuits present on the introduced nucleic acids. In some cases, the modified cell is modulated ex vivo. In other cases, the cell is introduced into (e.g., the individual from whom the cell was obtained) and/or already present in an individual; and the cell is modulated in vivo, e.g., by administering a nucleic acid or vector to the individual in vivo.

In some instances, cells employing a molecular circuit of the present disclosure may be therapeutic cells useful in cellular therapy of a subject. For example, in an application such as cellular therapy employing immune cells, the immune cells are engineered to deliver a therapeutic payload of interest in the human body. If the output of these engineered cells is too high, toxic effects may occur (such as e.g., cytokine release syndrome (CRS) as observed in CAR-T cell therapies), but on the other hand an output that is too low then the therapy may be ineffective. Therapeutic cells can be fine-tuned to achieve a desired level of output (i.e., a setpoint) under well-controlled laboratory conditions. However, the dynamic environments in which engineered therapeutic cells function make guaranteeing that the output will remain constant over time difficult. Using the molecular circuits described herein for implementing feedback control, engineered cells can automatically correct against disturbances encountered the environment, including e.g., disturbances that cause the output to drift. In one aspect, self-regulating engineered cells are more robust in in vivo scenarios, thus improving existing cell therapy applications of synthetic biology.

In some instances, cellular therapeutics such as CAR-T cells or synthetic receptor (e.g., SynNotch) enabled T cells greatly benefit from feedback control as a safety mechanism. A molecular circuit in a CAR-T cell may regulate the level of T cell activation and prevent toxic effects such as CRS which result from overstimulation of immune cells. Similarly, in SynNotch T cells, e.g., a molecular circuit may enable delivery of a precise concentration of a payload of interest regardless of any disturbances to the engineered cell that are present or introduced.

Circuits and/or methods of the present disclosure may be used in conjunction with several different production techniques known in the art, such as the production of biological products using cells in a bioreactor (e.g., mammalian, yeast, bacteria, and/or insect cells), methods involving the use of transgenic animals (e.g. goats or chickens), methods involving the use of transgenic plants (e.g., tobacco, seeds or moss), and other methods known to those of skill in the art.

Where employed, suitable cells for bioproduction may include but are not limited to e.g., COS cells, NS0 cells, SP2/0 cells, YB2/0 cells, and the like. Useful cells may be of prokaryotic (e.g., bacterial) or eukaryotic origin (including e.g., mammalian, yeast, plant, etc.) and may, in some instances, be established cell culture lines. Suitable cells may, in some instances, also include HeLa cells (e.g., American Type Culture Collection (ATCC) No. CCL-2), CHO cells (e.g., ATCC Nos. CRL9618, CCL61, CRL9096), 293 cells (e.g., ATCC No. CRL-1573), Vero cells, NIH 3T3 cells (e.g., ATCC No. CRL-1658), Huh-7 cells, BHK cells (e.g., ATCC No. CCL10), PC 12 cells (ATCC No. CRL1721), COS cells, COS-7 cells (ATCC No. CRL1651), RATI cells, mouse L cells (ATCC No. CCLI.3), human embryonic kidney (HEK) cells (ATCC No. CRL1573), HLHepG2 cells, and the like.

In some instances, useful bioproduction cells may include yeast cells. Suitable yeast cells include, but are not limited to, Pichia pastoris, Pichia finlandica, Pichia trehalophila, Pichia koclamae, Pichia membranaefaciens, Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Kluyveromyces sp., Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum, Neurospora crassa, Chlamydomonas reinhardtii, and the like.

In some instances, useful bioproduction cells may include prokaryotic cells. Suitable prokaryotic cells include, but are not limited to, any of a variety of laboratory strains of Escherichia coli, Lactobacillus sp., Salmonella sp., Shigella sp., and the like. See, e.g., Carrier et al. (1992) J. Immunol. 148:1176-1181; U.S. Pat. No. 6,447,784; and Sizemore et al. (1995) Science 270:299-302. Examples of Salmonella strains which can be employed include, but are not limited to, Salmonella typhi and S. typhimurium. Suitable Shigella strains include, but are not limited to, Shigella flexneri, Shigella sonnei, and Shigella disenteriae. Typically, the laboratory strain is one that is non-pathogenic. Non-limiting examples of other suitable bacteria include, but are not limited to, Bacillus subtilis, Pseudomonas pudita, Pseudomonas aeruginosa, Pseudomonas mevalonii, Rhodobacter sphaeroides, Rhodobacter capsulatus, Rhodospirillum rubrum, Rhodococcus sp., and the like. In some embodiments, the cell is Escherichia coli.

In some instances, molecular circuits are employed for metabolic engineering, where extended expression of an intermediate or constitutive expression of this intermediate without input is detrimental. It is common for intermediates or even final products of metabolic pathways to have at least some level of toxicity to the host cell. Therefore, optimization of their expression dynamics in pulses or only as certain other intermediate are at certain concertation levels is beneficial to maximizing the amount of product produced while maintaining effective cell growth.

Methods

The present disclosure also provides methods modulating a signaling pathway of a cell where the cell is or has been genetically modified with a molecular circuit disclosed herein.

In some embodiments, the method may comprise: exposing the cell to the first external stimulus, thereby activating expression the target protein of (a) and the inactivating protein of (b). In some embodiments, the cell further comprises a controller protein that controls the interaction between the inactivating protein of (b) and the target protein of (a), wherein expression and/or activity of the controller protein is modulated by a second exogenous stimulus, and the method further comprises exposing the cell to the second external stimulus.

The method may be done in vivo, ex vivo, or in vitro.

Methods employed for modulating signaling of a signaling pathway of a cell may serve various purposes. For example, in some instances, a circuit of the present disclosure may be employed in a method to provide control of a signaling pathway in response to an exogenous stimulus. In some instances, molecular circuit may include negative feedback control, which may, among other aspects, e.g., prevent the pathway from remaining active when a pathway output is produced and/or produced at or above a threshold level. In some instances, molecular circuit may include positive feedback control, which may, among other aspects, e.g., provide for amplification of a pathway output. In some instances, a molecular circuit may provide for more stable output of a signaling pathway, including e.g., where the signaling output of the pathway is insulated from variables such as but not limited to e.g., environmental factors and inputs.

Cells of the methods of the present disclosure may vary and may include in vitro and/or ex vivo cells genetically modified with one or more nucleic acids encoding one or more components of one or more circuits as described herein. In some instances, cells are primary cells obtained from a subject. In some instances, cells are obtained from a cell culture.

Accordingly, methods of the present disclosure may include obtaining cells used in the method, including where such cells are unmodified or have already been genetically modified to include a molecular circuit of the present disclosure. In some instances, methods of the present disclosure may include performing the genetic modification. In some instances, methods of the present disclosure may include collecting cells, including where cells are collected before and/or after genetic modification. Methods of collecting cells may vary and may include e.g., collecting cells from a cell culture, collecting a cellular sample from a subject that includes the cells of interest, and the like.

Once the molecular circuit is initiated and/or a cell containing the molecular circuit is delivered, modulation of the signaling pathway in accordance with the molecular circuit may not necessitate further manipulation, i.e., regulation of the signaling pathway by the molecular circuit may be essentially automatic.

Accordingly, in certain methods employing cells that contain a molecular circuit of the present disclosure, the cells may be administered to the subject and no further manipulation of the molecular circuit need be performed. For example, where a subject is treated with cells that contain a molecular circuit of the present disclosure, the treatment may include administering the cells to the subject, including where such administration is the sole intervention to treat the subject.

In such methods, cells that may be administered may include, but are not limited to e.g., immune cells. In such methods, the molecular circuit may be configured, in some instances, to modulate signaling of a native or synthetic signaling pathway of the immune cell, such as but not limited to e.g., an immune activation pathway or an immune suppression pathway. Non-limiting examples of suitable immune activation pathways, whether regulated by native or synthetic means, include cytokine signaling pathways, B cell receptor signaling pathways, T cell receptor signaling pathways, and the like. Non-limiting examples of suitable immune suppression pathways, whether regulated by native or synthetic means, include inhibitory immune checkpoint pathways, and the like.

Methods of the present disclosure may include administering to a subject the cells that express a therapeutic agent. Such cells may include a molecular circuit of the present disclosure and may or may not be immune cells. For example, in some instances, a method may include administering to a subject a non-immune cell that produces a therapeutic agent, either endogenously or heterologously, where production of the therapeutic is controlled, in whole or in part, by the molecular circuit. In some instances, a method may include administering to a subject an immune cell that produces a therapeutic agent, either endogenously or heterologously, where production of the therapeutic is controlled, in whole or in part, by the molecular circuit. Non-limiting examples of suitable encoded therapeutic agents, include but are not limited to e.g., hormones or components of hormone production pathways, such as insulins or a component of an insulin production pathway, estrogen/progesterone or a component of an estrogen/progesterone production pathway, testosterone or a component of an androgen production pathway, growth hormone or component of a growth hormone production pathway, or the like.

Such methods may be employed, in some instances, to treat a subject for a condition, including e.g., where the condition is a deficiency in a metabolic or a hormone. In such instances, the molecular circuit may be configured such that the output of the molecular circuit controls, in whole or in part, production and/or secretion of a metabolic or a hormone.

In some instances, the instant methods may include contacting a cell with one or more nucleic acids encoding a molecular circuit wherein such contacting is sufficient to introduce the nucleic acid(s) into the cell. Any convenient method of introducing nucleic acids into a cell may find use herein including but not limited viral transfection, electroporation, lipofection, bombardment, chemical transformation, use of a transducible carrier (e.g., a transducible carrier protein), and the like. Nucleic acids may be introduced into cells maintained or cultured in vitro or ex vivo. Nucleic acids may also be introduced into a cell in a living subject in vivo, e.g., through the use of one or more vectors (e.g., viral vectors) that deliver the nucleic acids into the cell without the need to isolate, culture or maintain the cells outside of the subject.

Any convenient method of delivering the circuit encoding components may find use in the subject methods. In some instances, the subject circuit may be delivered by administering to the subject a cell expressing the circuit. In some instances, the subject circuit may be delivered by administering to the subject a nucleic acid comprising one or more nucleotide sequences encoding the circuit. Administering to a subject a nucleic acid encoding the circuit may include administering to the subject a cell containing the nucleic acid where the nucleic acid may or may not yet be expressed. In some instances, administering to a subject a nucleic acid encoding the circuit may include administering to the subject a vector designed to deliver the nucleic acid to a cell.

The subject methods may include introducing into a subject in need thereof, cells that contain nucleic acid sequences encoding a therapeutic, the expression of which is controlled, at least in part by a molecular circuit. The therapeutic may be a therapeutic for the treatment of cancer. The introduced cells may be immune cells, including e.g., myeloid cells or lymphoid cells.

Non-limiting examples of cancers that may be treated include, e.g., Acute Lymphoblastic Leukemia (ALL), Acute Myeloid Leukemia (AML), Adrenocortical Carcinoma, AIDS-Related Cancers (e.g., Kaposi Sarcoma, Lymphoma, etc.), Anal Cancer, Appendix Cancer, Astrocytomas, Atypical Teratoid/Rhabdoid Tumor, Basal Cell Carcinoma, Bile Duct Cancer (Extrahepatic), Bladder Cancer, Bone Cancer (e.g., Ewing Sarcoma, Osteosarcoma and Malignant Fibrous Histiocytoma, etc.), Brain Stem Glioma, Brain Tumors (e.g., Astrocytomas, Central Nervous System Embryonal Tumors, Central Nervous System Germ Cell Tumors, Craniopharyngioma, Ependymoma, etc.), Breast Cancer (e.g., female breast cancer, male breast cancer, childhood breast cancer, etc.), Bronchial Tumors, Burkitt Lymphoma, Carcinoid Tumor (e.g., Childhood, Gastrointestinal, etc.), Carcinoma of Unknown Primary, Cardiac (Heart) Tumors, Central Nervous System (e.g., Atypical Teratoid/Rhabdoid Tumor, Embryonal Tumors, Germ Cell Tumor, Lymphoma, etc.), Cervical Cancer, Childhood Cancers, Chordoma, Chronic Lymphocytic Leukemia (CLL), Chronic Myelogenous Leukemia (CML), Chronic Myeloproliferative Neoplasms, Colon Cancer, Colorectal Cancer, Craniopharyngioma, Cutaneous T-Cell Lymphoma, Duct (e.g., Bile Duct, Extrahepatic, etc.), Ductal Carcinoma In Situ (DCIS), Embryonal Tumors, Endometrial Cancer, Ependymoma, Esophageal Cancer, Esthesioneuroblastoma, Ewing Sarcoma, Extracranial Germ Cell Tumor, Extragonadal Germ Cell Tumor, Extrahepatic Bile Duct Cancer, Eye Cancer (e.g., Intraocular Melanoma, Retinoblastoma, etc.), Fibrous Histiocytoma of Bone (e.g., Malignant, Osteosarcoma, ect.), Gallbladder Cancer, Gastric (Stomach) Cancer, Gastrointestinal Carcinoid Tumor, Gastrointestinal Stromal Tumors (GIST), Germ Cell Tumor (e.g., Extracranial, Extragonadal, Ovarian, Testicular, etc.), Gestational Trophoblastic Disease, Glioma, Hairy Cell Leukemia, Head and Neck Cancer, Heart Cancer, Hepatocellular (Liver) Cancer, Histiocytosis (e.g., Langerhans Cell, etc.), Hodgkin Lymphoma, Hypopharyngeal Cancer, Intraocular Melanoma, Islet Cell Tumors (e.g., Pancreatic Neuroendocrine Tumors, etc.), Kaposi Sarcoma, Kidney Cancer (e.g., Renal Cell, Wilms Tumor, Childhood Kidney Tumors, etc.), Langerhans Cell Histiocytosis, Laryngeal Cancer, Leukemia (e.g., Acute Lymphoblastic (ALL), Acute Myeloid (AML), Chronic Lymphocytic (CLL), Chronic Myelogenous (CML), Hairy Cell, etc.), Lip and Oral Cavity Cancer, Liver Cancer (Primary), Lobular Carcinoma In Situ (LCIS), Lung Cancer (e.g., Non-Small Cell, Small Cell, etc.), Lymphoma (e.g., AIDS-Related, Burkitt, Cutaneous T-Cell, Hodgkin, Non-Hodgkin, Primary Central Nervous System (CNS), etc.), Macroglobulinemia (e.g., Waldenstrom, etc.), Male Breast Cancer, Malignant Fibrous Histiocytoma of Bone and Osteosarcoma, Melanoma, Merkel Cell Carcinoma, Mesothelioma, Metastatic Squamous Neck Cancer with Occult Primary, Midline Tract Carcinoma Involving NUT Gene, Mouth Cancer, Multiple Endocrine Neoplasia Syndromes, Multiple Myeloma/Plasma Cell Neoplasm, Mycosis Fungoides, Myelodysplastic Syndromes, Myelodysplastic/Myeloproliferative Neoplasms, Myelogenous Leukemia (e.g., Chronic (CML), etc.), Myeloid Leukemia (e.g., Acute (AML), etc.), Myeloproliferative Neoplasms (e.g., Chronic, etc.), Nasal Cavity and Paranasal Sinus Cancer, Nasopharyngeal Cancer, Neuroblastoma, Non-Hodgkin Lymphoma, Non-Small Cell Lung Cancer, Oral Cancer, Oral Cavity Cancer (e.g., Lip, etc.), Oropharyngeal Cancer, Osteosarcoma and Malignant Fibrous Histiocytoma of Bone, Ovarian Cancer (e.g., Epithelial, Germ Cell Tumor, Low Malignant Potential Tumor, etc.), Pancreatic Cancer, Pancreatic Neuroendocrine Tumors (Islet Cell Tumors), Papillomatosis, Paraganglioma, Paranasal Sinus and Nasal Cavity Cancer, Parathyroid Cancer, Penile Cancer, Pharyngeal Cancer, Pheochromocytoma, Pituitary Tumor, Pleuropulmonary Blastoma, Primary Central Nervous System (CNS) Lymphoma, Prostate Cancer, Rectal Cancer, Renal Cell (Kidney) Cancer, Renal Pelvis and Ureter, Transitional Cell Cancer, Retinoblastoma, Rhabdomyosarcoma, Salivary Gland Cancer, Sarcoma (e.g., Ewing, Kaposi, Osteosarcoma, Rhabdomyosarcoma, Soft Tissue, Uterine, etc.), Sezary Syndrome, Skin Cancer (e.g., Childhood, Melanoma, Merkel Cell Carcinoma, Nonmelanoma, etc.), Small Cell Lung Cancer, Small Intestine Cancer, Soft Tissue Sarcoma, Squamous Cell Carcinoma, Squamous Neck Cancer (e.g., with Occult Primary, Metastatic, etc.), Stomach (Gastric) Cancer, T-Cell Lymphoma, Testicular Cancer, Throat Cancer, Thymoma and Thymic Carcinoma, Thyroid Cancer, Transitional Cell Cancer of the Renal Pelvis and Ureter, Ureter and Renal Pelvis Cancer, Urethral Cancer, Uterine Cancer (e.g., Endometrial, etc.), Uterine Sarcoma, Vaginal Cancer, Vulvar Cancer, Waldenstrom Macroglobulinemia, Wilms Tumor, and the like.

In some instances, methods of the present disclosure may be employed to treat a subject for an immune dysfunction, including but not limited to e.g., where the condition is an autoimmune disease. For example, in some instances, a molecular circuit of the present disclosure may be configured to regulate the immune activation level of a subject having an autoimmune disease, thus controlling the subject's autoimmune response to treat the subject for the autoimmune disease. In some instances, a subject having an autoimmune disease may be administered to cells configured to contain a molecular circuit of the present disclosure where the output of the molecular circuit is immune suppression.

The present disclosure further includes methods of making the nucleic acids, circuits, and cells employed in the herein described methods. In making the subject nucleic acids and circuits, and components thereof, any convenient methods of nucleic acid manipulation, modification and amplification (e.g., collectively referred to as “cloning”) may be employed. In making the subject cells, containing the nucleic acids encoding the described circuits, convenient methods of transfection, transduction, culture, etc., may be employed.

A nucleotide sequence encoding all or a portion of the components of a circuit of the present disclosure can be present in an expression vector and/or a cloning vector. Where a subject circuit or component thereof is split between two or more separate polypeptides, nucleotide sequences encoding the two or more polypeptides can be cloned in the same or separate vectors. An expression vector can include a selectable marker, an origin of replication, and other features that provide for replication and/or maintenance of the vector. Suitable expression vectors include, e.g., plasmids, viral vectors, and the like.

Large numbers of suitable vectors and promoters are known to those of skill in the art; many are commercially available for generating a subject recombinant construct. The following vectors are provided by way of example. Bacterial: pBs, phagescript, PsiX174, pBluescript SK, pBs KS, pNH8a, pNH16a, pNH18a, pNH46a (Stratagene, La Jolla, Calif., USA); pTrc99A, pKK223-3, pKK233-3, pDR540, and pRTT5 (Pharmacia, Uppsala, Sweden). Eukaryotic: pWLneo, pSV2cat, pOG44, PXR1, pSG (Stratagene) pSVK3, pBPV, pMSG and pSVL.

Expression vectors generally have convenient restriction sites located near the promoter sequence to provide for the insertion of nucleic acid sequences encoding heterologous proteins. A selectable marker operative in the expression host may be present. Suitable expression vectors include, but are not limited to, viral vectors (e.g. viral vectors based on vaccinia virus; poliovirus; adenovirus (see, e.g., Li et ah, Invest Opthalmol Vis Sci 35:2543 2549, 1994; Borras et ah, Gene Ther 6:515 524, 1999; Li and Davidson, PNAS 92:7700 7704, 1995; Sakamoto et ah, H Gene Ther 5:1088 1097, 1999; WO 94/12649, WO 93/03769; WO 93/19191; WO 94/28938; WO 95/11984 and WO 95/00655); adeno-associated virus (see, e.g., Ali et ah, Hum Gene Ther 9:81 86, 1998, Flannery et ah, PNAS 94:6916 6921, 1997; Bennett et ah, Invest Opthalmol Vis Sci 38:2857 2863, 1997; Jomary et ah, Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et ah, J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, human immunodeficiency virus, myeloproliferative sarcoma virus, and mammary tumor virus); and the like.

As noted above, in some embodiments, a nucleic acid comprising a nucleotide sequence encoding a molecular circuit or component thereof of the present disclosure will in some embodiments be DNA or RNA, e.g., in vitro synthesized DNA, recombinant DNA, in vitro synthesized RNA, recombinant RNA, etc. Methods for in vitro synthesis of DNA/RNA are known in the art; any known method can be used to synthesize DNA/RNA comprising a desired sequence. Methods for introducing DNA/RNA into a host cell are known in the art. Introducing DNA/RNA into a host cell can be carried out in vitro or ex vivo or in vivo. For example, a host cell (e.g., an NK cell, a cytotoxic T lymphocyte, etc.) can be transduced, transfected or electroporated in vitro or ex vivo with DNA/RNA comprising a nucleotide sequence encoding all or a portion of a circuit of the present disclosure.

Methods of the instant disclosure may further include culturing a cell genetically modified to encode a molecular circuit of the instant disclosure including but not limited to e.g., culturing the cell prior to administration, culturing the cell in vitro or ex vivo (e.g., the presence or absence of one or more antigens), etc. Any convenient method of cell culture may be employed whereas such methods will vary based on various factors including but not limited to e.g., the type of cell being cultured, the intended use of the cell (e.g., whether the cell is cultured for research or therapeutic purposes), etc. In some instances, methods of the instant disclosure may further include common processes of cell culture including but not limited to e.g., seeding cell cultures, feeding cell cultures, passaging cell cultures, splitting cell cultures, analyzing cell cultures, treating cell cultures with a drug, harvesting cell cultures, etc.

Methods of the instant disclosure may, in some instances, further include receiving and/or collecting cells that are used in the subject methods. In some instances, cells are collected from a subject. Collecting cells from a subject may include obtaining a tissue sample from the subject and enriching, isolating and/or propagating the cells from the tissue sample. Isolation and/or enrichment of cells may be performed using any convenient method including e.g., isolation/enrichment by culture (e.g., adherent culture, suspension culture, etc.), cell sorting (e.g., FACS, microfluidics, etc.), and the like. Cells may be collected from any convenient cellular tissue sample including but not limited to e.g., blood (including e.g., peripheral blood, cord blood, etc.), bone marrow, a biopsy, a skin sample, a cheek swab, etc. In some instances, cells are received from a source including e.g., a blood bank, tissue bank, etc. Received cells may have been previously isolated or may be received as part of a tissue sample thus isolation/enrichment may be performed after receiving the cells and prior to use. In certain instances, received cells may be non-primary cells including e.g., cells of a cultured cell line. Suitable cells for use in the herein described methods are further detailed herein.

Kits

Certain aspects of the present disclosure also include kits. The kits may include, e.g., one or more of any of the reaction mixture components described above with respect to the subject methods. For example, the kits may include a nucleic acid encoding a molecular circuit, components for delivery, cloning and/or expression, and the like, in various combinations.

Components of the kits may be present in separate containers, or multiple components may be present in a single container. In some instances, components of the subject kits may be presented as a “cocktail” where, as used herein, a cocktail refers to a collection or combination of two or more different but similar components in a single vessel.

Certain embodiments of the invention provide a cell comprising a molecular circuit, the molecular circuit comprising:
(a) a first nucleic acid comprising a first nucleotide sequence encoding an inactivating protein that is activated by a first exogenous stimulus,
(b) a second nucleic acid comprising a second nucleotide sequence encoding a target protein that is activated by the first exogenous stimulus, wherein the target protein has a regulatory motif and the activated inactivating protein inactivates the target protein via the regulatory motif.

In certain cases, upon exposure of the cell to the first exogenous stimulus, the activation of the inactivating protein is delayed relative to the activation of the target protein.

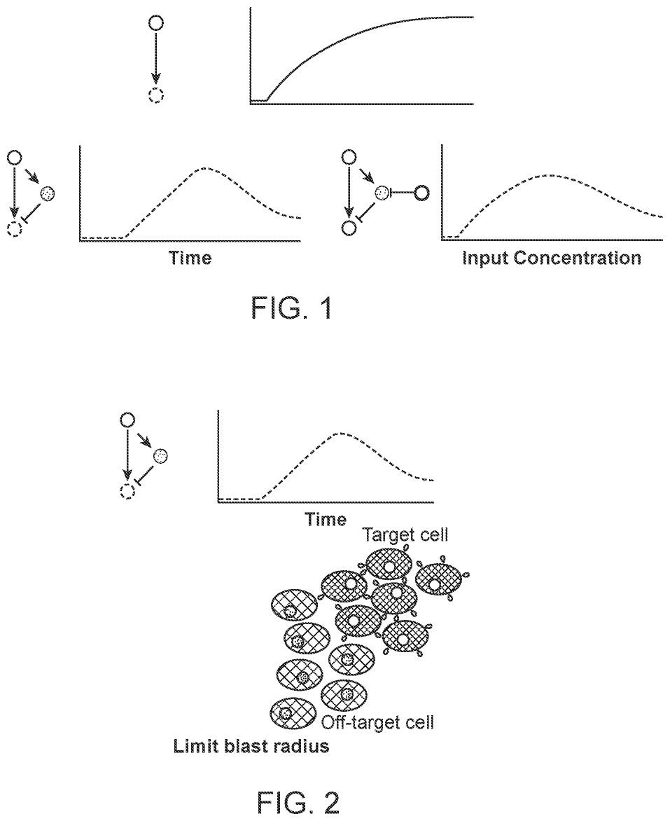

An example of such molecular circuit is described in FIG. 1, bottom left panel. The orange circle represents a cell's response to a first exogenous stimulus, which induces a target protein, indicated by the blue circle, and an inactivating protein, indicated by the green circle. The inactivating protein inhibits the target protein, thereby producing a pulse of the response to the stimulus, as indicated in the graph of time versus response.

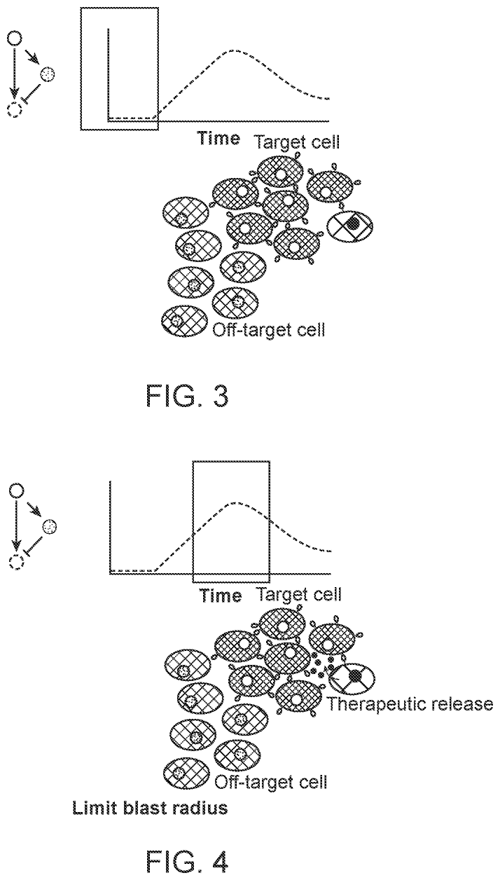

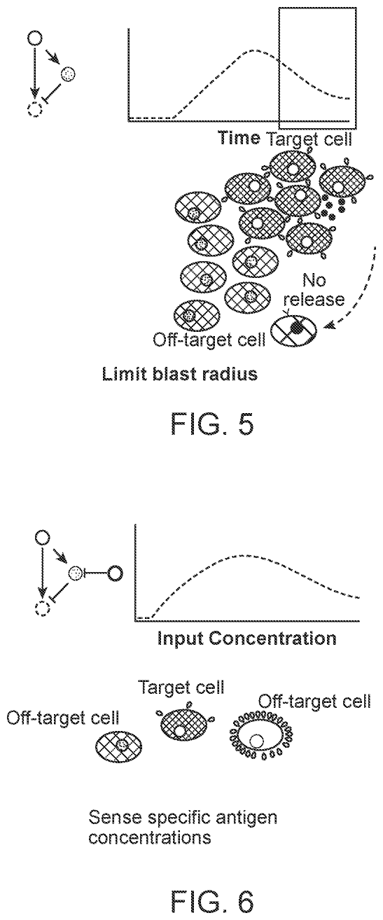

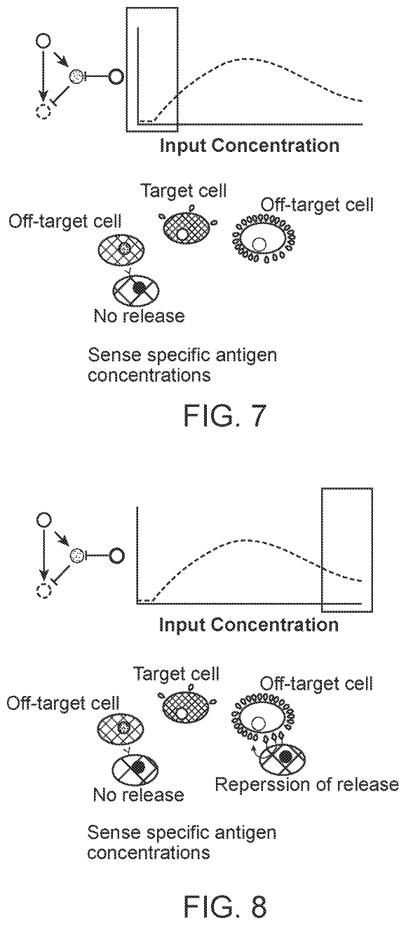

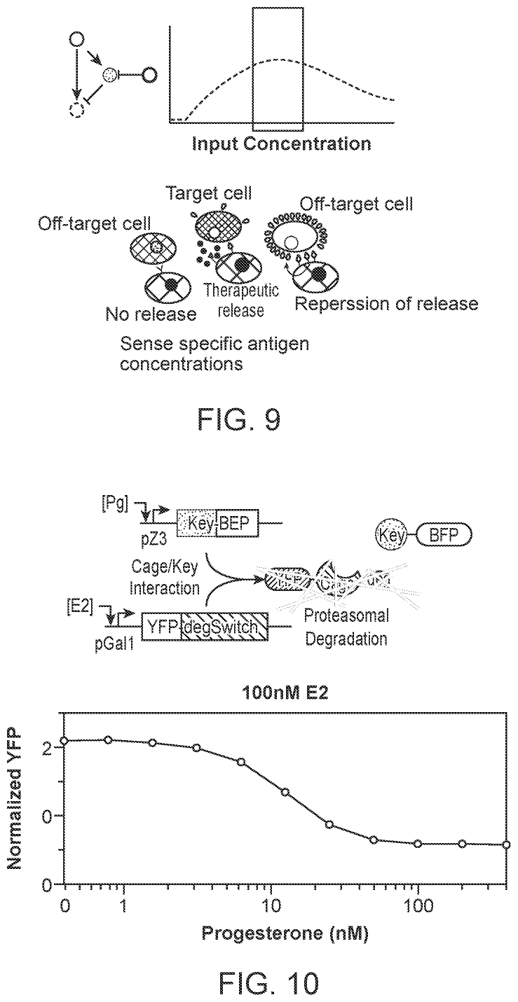

FIGS. 2-5 provide a “pulse generator” molecular circuit used as a delayed off switch to reduce off target effects in cell-based therapies.

The first exogenous stimulus can activate the target protein and the inactivating protein by inducing the expression of the target protein and the inactivating protein. In certain cases, the first exogenous stimulus induces the expression of the target protein and, after a delay, the first exogenous stimulus induces the expression of the inactivating protein. For example, the first exogenous stimulus:
i) induces the expression of the target protein via a signaling pathway; and
ii) induces, via the signaling pathway and one or more further signaling steps, the expression of the inactivating protein.

Accordingly, a cell can comprise:
a) the first nucleotide sequence encoding the inactivating protein operably connected to a first heterologous promoter that is responsive to the first exogenous stimulus via the second transcriptional signal, and
b) the second nucleotide sequence encoding the target protein operably connected to a second heterologous promoter that is responsive to the first exogenous stimulus via the first transcription signal.

In certain cases, the first exogenous stimulus initiates a signaling pathway that can induce the expression of the target protein via the activation of a first transcription signal that induces the expression of the target protein. The first transcription signal can comprise a first set of one or more transcription factors that are induced by the first exogenous stimulus. The first set of transcription factors can then bind to the regulatory sequence in the second nucleic acid to induce the expression of the target protein.

In certain embodiments, the delay in the activation of the inactivating protein relative to the activation of the target protein is provided by the intermediate activation of the first transcription signal. For example, the first exogenous stimulus can initiate a signaling pathway that can activate a first transcription signal that induces the expression of the target protein and the first transcription signal also induces a second transcription signal via one or more proteins, wherein the second transcription signal induces the expression of the inactivating protein.

The first transcription signal can be a first set of transcription factors that are activated by the signaling pathway, wherein the first set of transcription factors induce transcription of the target protein. Similarly, the second transcription signal comprises a second set of transcription factors, wherein the first transcription signal induces the expression of the second set of transcription factors, and wherein the second set of transcription factors induce the transcription of the inactivating protein.

In certain cases, the inactivating protein inactivates the target protein by inducing degradation of the target protein via the regulatory motif. For example, the regulatory motif of the target protein can comprise a degron and the inactivating protein can comprise a key. The key can induce degradation of the target protein via the degron by exposing the degradation sequence in the degron.

In certain cases, the inactivating protein inactivates the target protein by inducing translocation of the target protein to another subcellular compartment via the regulatory motif. For example, the regulatory motif of the target protein can comprise a subcellular localization signal and the inactivating protein can comprise a key. The key can induce translocation of the target protein by exposing the subcellular localization signal in the regulatory motif.

The activated inactivating protein can directly inactivate the target protein via the regulatory motif. The activated inactivating protein can also indirectly inactivate the target protein via the regulatory motif.

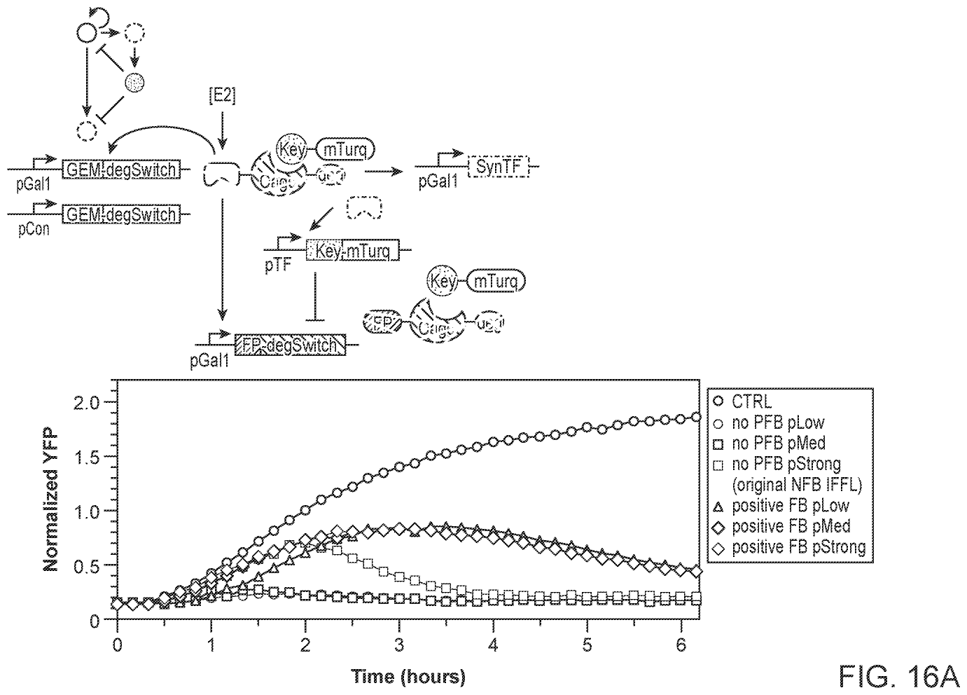

An example of a molecular circuit is provided in FIG. 12. A first exogenous stimulus initiates the expression of a transcription factor (GEM) that induces the expression of a target protein (FP-degSwitch). The same transcription factor, GEM, also induces the expression of another transcription factor (SynTF), which in turn induces the expression of an inactivating protein (Key-mTurq). The inactivating protein, when expressed, interacts with the degSwitch of the target protein to induce degradation of the target protein.

Certain molecular circuits disclosed herein can be used to control a cell's response to an exogenous stimulus such that the cell responds to the stimulus only up to a predetermined strength of the stimulus and does not respond to the stimulus beyond the predetermined strength. Thus, these circuits can be used to control a cell's response so that the cell responds to a stimulus of only up to a particular strength of a stimulus. Such control is achieved by interaction between a first stimulus and a second stimulus, wherein the cell's response to the second stimulus controls the cell's response to the first stimulus.

Thus, certain embodiments of the invention provide a cell comprising a molecular circuit, the molecular circuit comprising:
(a) a first nucleic acid comprising a first nucleotide sequence encoding an inactivating protein that is activated by a first exogenous stimulus,
(b) a second nucleic acid comprising a second nucleotide sequence encoding a target protein that is activated by the first exogenous stimulus, wherein the target protein has a regulatory motif and the activated inactivating protein inactivates the target protein via the regulatory motif, and
(c) a third nucleic acid comprising a third nucleotide sequence encoding a controller protein that is activated by a second exogenous stimulus.

The controller protein controls the interaction between the inactivating protein and the target protein. For example, the controller protein can inhibit the inactivating protein thereby relieving the inactivating protein mediated inhibition of the target protein. The inhibition of an inactivating protein by a controller protein can be mediated by inducing degradation or translocation of the inactivating protein mediated by such interaction. The inhibition of an inactivating protein by a controller protein can be mediated by controller protein mediate interference between the interaction of the inactivating protein and target protein.

Accordingly, a cell can comprise:
a) the first nucleotide sequence encoding the inactivating protein operably connected to a first heterologous promoter that is responsive to the first exogenous stimulus,
b) the second nucleotide sequence encoding the target protein operably connected to a second heterologous promoter that is responsive to the first exogenous stimulus, and
c) the third nucleotide sequence encoding the controller protein operably connected to a third heterologous promoter that is responsive to the second exogenous stimulus.

Certain embodiments of the disclosure provide a concentration filter molecular circuit having the expression level of the controller protein as an integral part of the cell, i.e., where the controller protein is constitutively expressed. In certain embodiments, the expression level of the controller protein can be under the control of a constitutive promoter and the strength of the constitutive promoter can be used to determine the threshold of the first exogenous stimulus. For example, a controller protein can be expressed under the control of a promoter whose strength corresponds to a certain strength of a second exogenous stimulus. Thus, the expression level of the controller protein under the control of the constitutive promoter can be the expression level of the controller protein under a certain strength of the second exogenous stimulus. Therefore, if a desirable threshold of the first exogenous stimulus can be controlled by the expression level of a controller protein under a certain strength of a second exogenous stimulus, then a cell can be produced having the controller protein under the control of a constitutive promoter that produces the expression level of the controller protein produced under that strength of the exogenous stimulus. Such molecular circuits require fewer genetic modifications to produce a concentration filter molecular circuit relative to the circuits having the expression of the controller protein under the second exogenous stimulus.

Accordingly, certain embodiments of the invention provide a cell comprising a molecular circuit, the molecular circuit comprising:
(a) a first nucleic acid comprising a first nucleotide sequence encoding an inactivating protein that is activated by a first exogenous stimulus,
(b) a second nucleic acid comprising a second nucleotide sequence encoding a target protein that is activated by the first exogenous stimulus, wherein the target protein has a regulatory motif and the activated inactivating protein inactivates the target protein via the regulatory motif, and
(c) a third nucleic acid comprising a third nucleotide sequence encoding a controller protein at a predetermined level.

Accordingly, a cell can comprise:
a) the first nucleotide sequence encoding the inactivating protein operably connected to a first heterologous promoter that is responsive to the first exogenous stimulus,
b) the second nucleotide sequence encoding the target protein operably connected to a second heterologous promoter that is responsive to the first exogenous stimulus, and
c) the third nucleotide sequence encoding the controller protein operably connected to a third heterologous promoter that constitutively induces the expression of the controller protein at the predetermined level.

In certain cases, the first exogenous stimulus initiates a signaling pathway that can induce the expression of the target protein and/or the inactivating protein via the activation of a first transcription signal that induces the expression of these proteins. The first transcription signal can comprise a first set of one or more transcription factors that are induced by the first exogenous stimulus. The first set of transcription factors can then bind to the regulatory sequences in the first and second nucleic acids to induce the expression of the target protein and the inactivating protein.

Similarly, the second exogenous stimulus initiates a signaling pathway that can induce the expression of the controller protein via the activation of a second transcription signal that induces the expression of the controller protein. The second transcription signal can comprise a second set of one or more transcription factors that are induced by the second exogenous stimulus. The second set of transcription factors can then bind to the regulatory sequences in the first and second nucleic acids to induce the expression of the controller protein.

Unlike the pulse generator circuit, certain embodiments of the concentration filter circuit do not comprise a delay in the activation of the inactivating protein relative to the activation of the target protein. Therefore, the first exogenous stimulus can initiate a signaling pathway that can activate a first transcription signal that induces the expression both the target protein and the inactivating protein.

In certain cases, the inactivating protein inactivates the target protein by inducing degradation of the target protein via the regulatory motif. For example, the regulatory motif of the target protein can comprise a degron and the inactivating protein can comprise a key. The key can induce degradation of the target protein via the degron by exposing the degradation sequence in the degron.

In certain cases, the inactivating protein inactivates the target protein by inducing translocation of the target protein to another subcellular compartment via the regulatory motif. For example, the regulatory motif of the target protein can comprise a subcellular localization signal and the inactivating protein can comprise a key. The key can induce translocation of the target protein by exposing the subcellular localization signal in the regulatory motif.

The activated inactivating protein can directly inactivate the target protein via the regulatory motif. The activated inactivating protein can also indirectly inactivate the target protein via the regulatory motif.

In certain such embodiments, the controller protein is activated by a second exogenous stimulus, wherein the controller protein controls the interactions between the inactivating protein and the target protein. The controller protein can relieve the inactivating protein mediated inhibition of the target protein, thereby allowing the cell to activate the target protein in response to the first stimulus. When the strength of the first stimulus exceeds a threshold, the activation of the inactivating protein is sufficiently strong so that the controller protein cannot relieve the inactivating protein mediated inhibition of the target protein and the inactivating protein inhibits the target protein. Thus, the cell does not activate the target protein in response to the first stimulus when the strength of the first stimulus exceeds the threshold. Thus, the relative strengths of the first and the second stimuli and the cell's response to these stimuli provide the cell the ability to respond to the first exogenous stimulus only up to a threshold strength of the first exogenous stimulus.

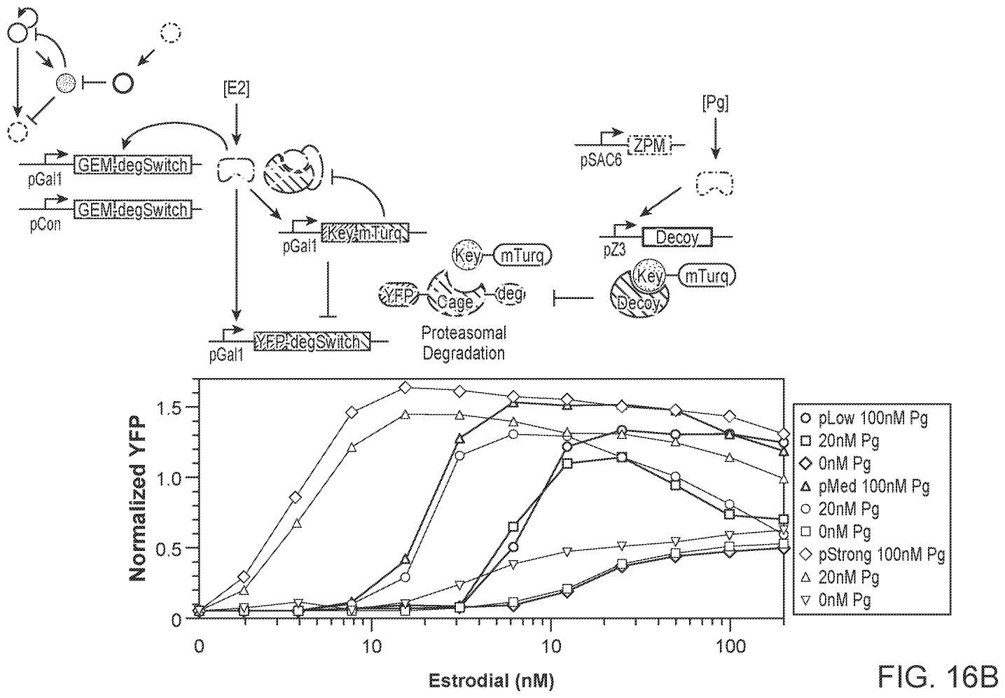

An example of such molecular circuit is provided in FIG. 13. In FIG. 13, a first exogenous stimulus [E2] induces a transcription factor (GEM), which induces the expression of the target protein (YFP-degSwitch) as well as the expression of inactivating protein (Key-mTurq). In the same cell, a second stimulus [Pg] induces the expression of a second transcription factor (ZPM), which in turn induces the expression of a decoy. The decoy sequesters the inactivating protein by binding to the key, thereby inhibiting the interaction between the key and the degSwitch of the target protein. If the strength of first exogenous stimulus [E2] is below a threshold, the decoy inhibits the key, thereby maintaining the expression of the target protein. However, if the strength of the first exogenous stimulus [E2] exceeds a threshold, the expression of the inactivating protein is increased via GEM and such increase overcomes the inhibition caused by the decoy. Once such inhibition is overcome, the key induces the degradation of the target protein via the interaction between the key and the degSwitch. Thus, such cell only produces high levels of target protein when the strength of the first exogenous stimulus is below a threshold.

Such threshold can be controlled by the strength of the second stimulus and/or the strength of the molecular pathway induced by the second stimulus. For example, higher binding affinity between the decoy and the key would induce a higher threshold strength of the first exogenous stimulus that would cause the expression of the target protein. In other words, higher strength of the first exogenous stimulus would be required for the first exogenous stimulus to induce sufficient activation of the inactivating protein, which would then inhibit the target protein.

In certain embodiments, positive and/or negative feedback mechanisms can be introduced between the inactivating proteins, target protein, the controller proteins, and/or the transcription signals that induce the expression of these proteins.

For example, a negative feedback can be introduced, where the inactivating protein inhibits the response of the cell to the first exogenous stimulus, such as the transcription signal induced by the first exogenous stimulus that induces the expression of the target protein and the inactivating protein. For example, a transcription factor induced by the first exogenous stimulus may contain a degron or a subcellular localization signal, which when exposed by the key in the inactivating protein, can induce the inactivation of the transcription factor, for example, via degradation or translocation of the transcription factor.

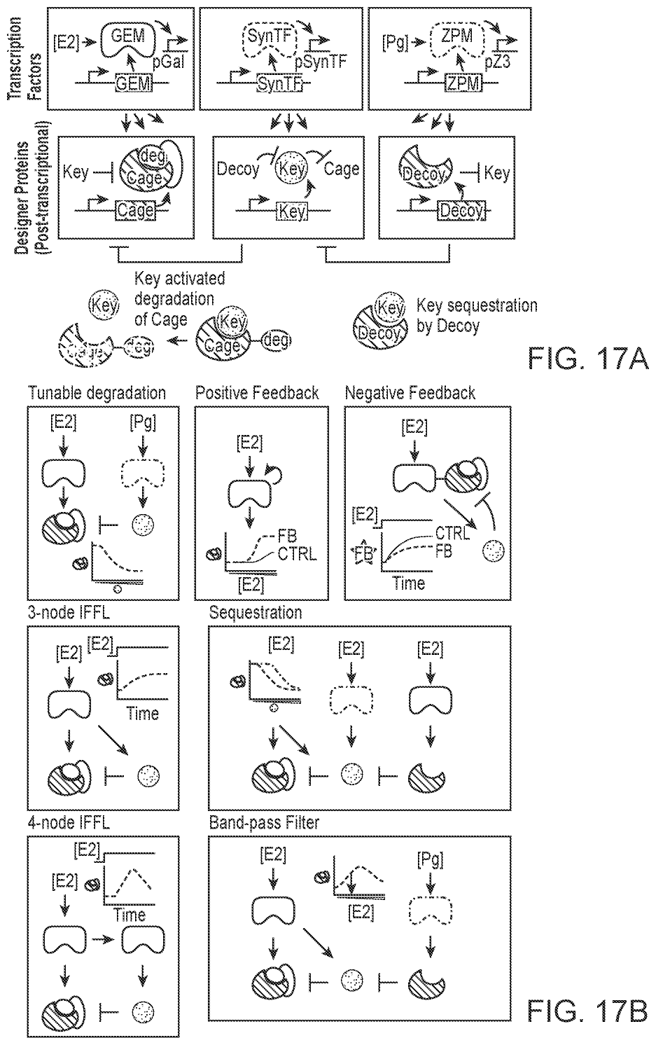

Negative feedback can reduce the steady-state output from the pulse generator circuit and further dampen responses from the concentration filter circuit at high inducer concentrations. Examples of molecular circuits comprising such negative feedback in pulse generator circuit are disclosed in FIG. 14A. Examples of molecular circuits comprising such negative feedback in concentration filter circuit are disclosed in FIG. 14B.

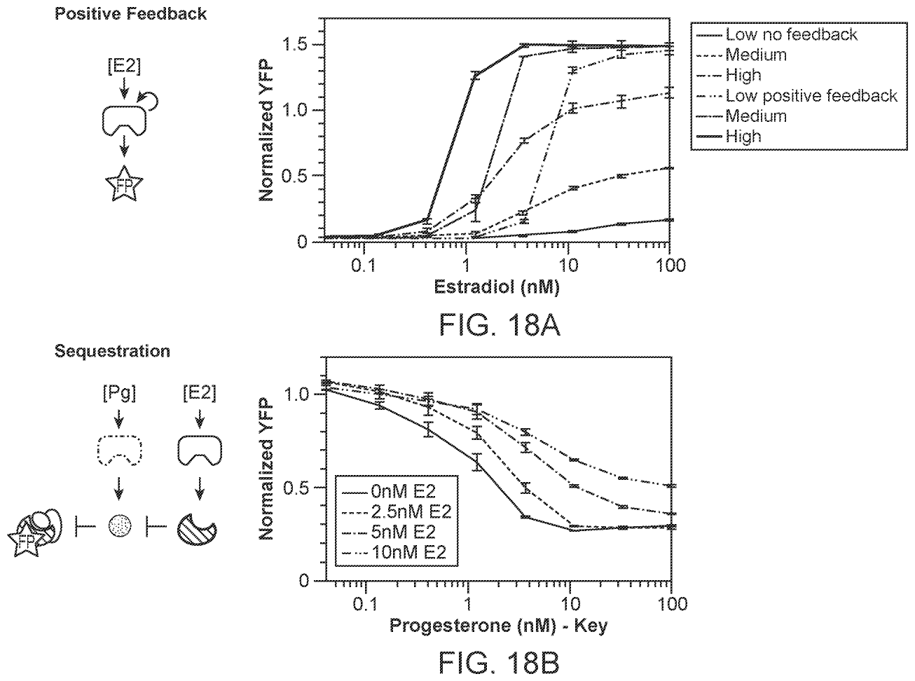

A positive feedback could be introduced, where the cell's response to the first exogenous stimulus further amplifies the cell's response of to the first exogenous stimulus. For example, a transcription signal produced in response to the first exogenous stimulus can further amplify itself to increase the transcription signal. In certain such embodiments, the first exogenous stimulus produces a transcription factor under the control of a promoter that is responsive to that transcription factor. Therefore, increased expression of the transcription factor in response to the first exogenous stimulus further stimulates the expression of the transcription factor because the transcription factor produced in response to the first exogenous stimulus further induces the transcription of additional molecules of that transcription factor. Typically, a transcription factor gene under the positive feedback control is provided as an additional copy of the gene under the control of a promoter that is responsive to the transcription factor. Thus, the initial expression of the transcription factor in response to the first exogenous stimulus may be under the control of a different transcription factor. Positive feedback can lengthen the duration of the pulse in the pulse generator and tune the threshold of activation of the concentration filter circuit. Examples of molecular circuits comprising such positive feedback in pulse generator circuit are disclosed in FIG. 15A. Examples of molecular circuits comprising such positive feedback in concentration filter circuit are disclosed in FIG. 15B.

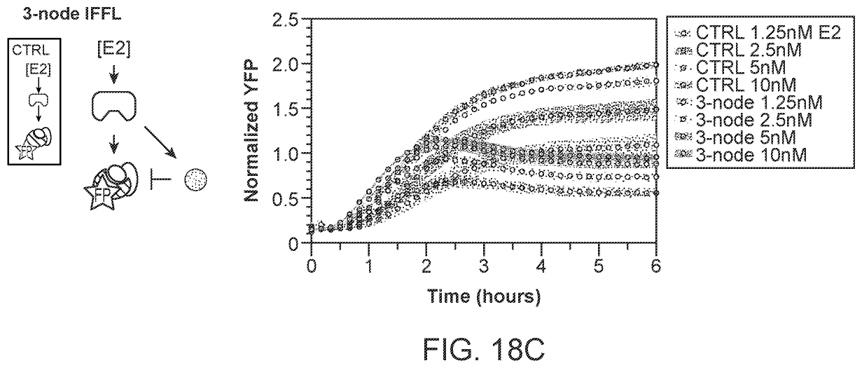

In certain cases, positive and negative feedback mechanisms could be introduced between the inactivating proteins, target protein, the controller proteins, and/or the transcription signals that induce the expression of these proteins. For example, a molecular circuit can comprise a negative feedback between the inactivating protein and the response of the cell to the first exogenous stimulus as well as a positive feedback in the cell's response to the first exogenous stimulus. Particularly, a molecular circuit can comprise both the negative and positive feedback described in the two preceding paragraphs or in FIGS. 14A-14B and 15A-15B. Examples of molecular circuits comprising a negative feedback and a positive feedback in pulse generator circuit are described in FIG. 16A. Examples of molecular circuits comprising a negative feedback and a positive feedback in concentration filter circuit are disclosed in FIG. 16B.

Certain embodiments of the invention provide a method of regulating the activity of a target protein in a cell by providing in the cell a molecular circuit disclosed herein and exposing the cell to a first exogenous stimulus and, optionally, to a second exogenous stimulus.

Further embodiments of the invention provide a method of treating a disease in a subject, comprising administering to the subject a cell comprising a molecular circuit disclosed herein. In certain such methods the disease comprises the presence in the subject of pathogenic cells that cause the disease in the subject and the pathogenic cells present the first exogenous stimulus to the cells. For example, the pathogenic cells could be cancerous cells and the first exogenous stimulus could be the interactions of the cancerous cells to the cells comprising the molecular circuits disclosed herein.

For example, the cell can be a T-cell that specifically binds to a cancer cell antigen and induces the killing of the cancer cell. Thus, the first exogenous stimulus comprises binding of the T-cell to the cancer antigen. Accordingly, the first exogenous stimulus could be the binding of the cell comprising the molecular circuit disclosed herein to the cancer specific antigens expressed on the cancerous cells.

In certain embodiments, the activity of the cells comprising molecular circuits disclosed herein can be controlled by a second exogenous stimulus presented by the pathogenic cells. Such second exogenous stimulus can comprise binding of the cells comprising molecular circuits disclosed herein to a house-keeping receptor. A “house-keeping receptor” is a receptor that is expressed in substantially equal quantities on the surface of the cancerous cells and the corresponding non-cancerous cells.

### EMBODIMENTS

In the following description, the term “signaling protein” is synonymous with the term “target protein” as used elsewhere in the specification, and the term “regulatory protein” is synonymous with the term “inactivating protein” as used elsewhere in the specification.

Embodiment 1. A cell comprising a molecular circuit, the molecular circuit comprising:
(a) a first nucleic acid comprising a first nucleotide sequence encoding a regulatory protein that is activated by a first exogenous stimulus, and
(b) a second nucleic acid comprising a second nucleotide sequence encoding a signaling protein that is activated by the first exogenous stimulus,
   wherein the signaling protein has a regulatory motif and the activated regulatory protein inactivates the signaling protein via the regulatory motif.

Embodiment 2. The cell according to embodiment 1, wherein upon exposure of the cell to the first exogenous stimulus, the activation of the regulatory protein is delayed relative to the activation of the signaling protein.

Embodiment 3. The cell of embodiment 1 or 2, wherein the first exogenous stimulus activates the signaling protein and the regulatory protein by inducing the expression of the signaling protein and the regulatory protein, wherein the first exogenous stimulus induces the expression of the signaling protein and, after a delay, the first exogenous stimulus induces the expression of the regulatory protein, wherein the first exogenous stimulus:
i) induces the expression of the signaling protein via a signaling pathway; and
ii) induces, via the signaling pathway and one or more further signaling steps, the expression of the regulatory protein.

Embodiment 4. The cell of embodiment 3, wherein:
i) the signaling pathway comprises activation of a first transcription signal and the first transcription signal induces the expression of the signaling protein; and
ii) the first transcription signal also induces the expression of one or more proteins that induce the one or more further signaling steps comprising a second transcription signal, and wherein the second transcription signal induces the expression of the regulatory protein.

Embodiment 5. The cell of embodiment 4, wherein:
i) the first transcription signal comprises a first set of transcription factors that are activated by the signaling pathway, wherein the first set of transcription factors induce transcription of the signaling protein; and
ii) the second transcription signal comprises a second set of transcription factors, wherein the first transcription signal induces the expression of the second set of transcription factors, and wherein the second set of transcription factors induce the transcription of the regulatory protein.

Embodiment 6. The cell of any of preceding embodiments, wherein the regulatory protein inactivates the signaling protein by inducing degradation of the signaling protein via the regulatory motif.

Embodiment 7. The cell of embodiment 6, wherein the regulatory motif of the signaling protein comprises a degron and the regulatory protein comprises a key, wherein the key induces degradation of the signaling protein via the degron.

Embodiment 8. The cell of any of embodiments 1 to 5, wherein the regulatory protein inactivates the signaling protein by inducing translocation of the signaling protein to a different subcellular location via the regulatory motif.

Embodiment 9. The cell of embodiment 8, wherein the regulatory motif of the signaling protein comprises a subcellular localization signal and the regulatory protein comprises a key, wherein the key induces translocation of the signaling protein to the different subcellular location via the subcellular localization signal.

Embodiment 10. The cell of any of preceding embodiments, wherein the activated regulatory protein directly inactivates the signaling protein via the regulatory motif.

Embodiment 11. The cell of any of embodiments 1 to 9, wherein the activated regulatory protein indirectly inactivates the signaling protein via the regulatory motif.

Embodiment 12. The cell of any of the preceding embodiments, further comprising a controller protein that controls the interaction between the regulatory protein and the signaling protein.

Embodiment 13. The cell of embodiment 12, wherein the controller protein controls the interaction between the regulatory protein and the signaling protein by binding to or inactivating the regulatory protein or blocking the interaction between the signaling protein and regulatory protein.

Embodiment 14. The cell of any of preceding embodiments, wherein:
a) the first nucleotide sequence encoding the regulatory protein is operably connected to a first heterologous promoter that is responsive to the first exogenous stimulus,
b) the second nucleotide sequence encoding the signaling protein is operably connected to a second heterologous promoter that is responsive to the first exogenous stimulus, and
c) when present, a third nucleotide sequence encoding the controller protein is operably connected to a third heterologous promoter that is responsive to the second exogenous stimulus.

Embodiment 15. The cell of any of preceding embodiments, wherein the cell is a T-cell and the first exogenous stimulus comprises binding of the T-cell to a cancer antigen.

Embodiment 16. The cell according any of the preceding embodiments, wherein the regulatory protein inhibits the response of the cell to the first exogenous stimulus.

Embodiment 17. The cell of embodiment 16, wherein the response of the cell to the first exogenous stimulus comprises induction of the first transcription signal.

Embodiment 18. The cell according to any of the preceding embodiments, wherein the response of the cell to the first exogenous stimulus further amplifies the response of the cell to the first exogenous stimulus.

Embodiment 19. The cell according to embodiment 18, wherein the response of the cell to the first exogenous stimulus comprises induction of the first transcription signal.

Embodiment 20. The cell according any of the preceding embodiments, wherein the regulatory protein inhibits the response of the cell to the first exogenous stimulus and the response of the cell to the first exogenous stimulus further amplifies the response of the cell to the first exogenous stimulus.

Embodiment 21. The cell of embodiment 20, wherein the response of the cell to the first exogenous stimulus comprises induction of the first transcription signal.

Embodiment 22. A method of regulating the activity of the signaling protein in the cell of any of preceding embodiments, the method comprising exposing the cell to the first exogenous stimulus.

Embodiment 23. The method of embodiment 22, further comprising exposing the cell to the second exogenous stimulus.

Embodiment 24. A method of treating a disease in a subject, comprising administering to the subject the cell of any of embodiments 1 to 21, wherein the disease comprises the presence in the subject of pathogenic cells that cause the disease in the subject, wherein the pathogenic cells present the first exogenous stimulus to the cells.

Embodiment 25. The method of embodiment 24, wherein, in addition to the first exogenous stimulus, the pathogenic cells also present the second exogenous stimulus to the cells.

Embodiment 26. The method of embodiment 24 or 25, wherein the disease is a cancer and the pathogenic cells are cancerous cells in the subject.

Embodiment 27. The method of embodiment 26, wherein the first exogenous stimulus presented by the pathogenic cells comprises a cancer antigen.

Embodiment 28. The method of embodiment 26 or 27, wherein the second exogenous stimulus presented by the pathogenic cells comprises a house-keeping receptor.

All patents, patent applications, provisional applications, and publications referred to or cited herein are incorporated by reference in their entirety, including all figures and tables, to the extent they are not inconsistent with the explicit teachings of this specification.

Following are examples which illustrate procedures for practicing the invention. These examples should not be construed as limiting. All percentages are by weight and all solvent mixture proportions are by volume unless otherwise noted.

### Examples I

Using a combination of synthetic transcription factors and designer proteins to activate and block degradation, a library of pulse generators and band-pass filters in yeast was constructed. The designs for pulse generators and filters build on a type 1 incoherent feed forward loop (I1-FFL) with additional positive and negative feedback that tune its response. The I1-FFL can produce a pulse of gene expression. I1-FFLs can aid implementation of band-pass filters, fold-change detection, biosensing, and noise buffering.

Here, various circuit topologies were designed, built, and tested to demonstrate a range of signal processing capabilities. For example, the behavior of a few simple networks-positive feedback, sequestration, and a 3-node I1-FFL was examined. A 4-node I1-FFL was used to generate a pulse. Negative feedback was layered on the 4-node I1-FFL to reduce the steady state gene expression and positive feedback to increase the pulse height and duration. In addition, filtering was demonstrated by combining the 3-node I1-FFL and sequestration. Positive and negative feedback was layered on the filter to shift and amplify the circuit response and reduce the high input response respectively.

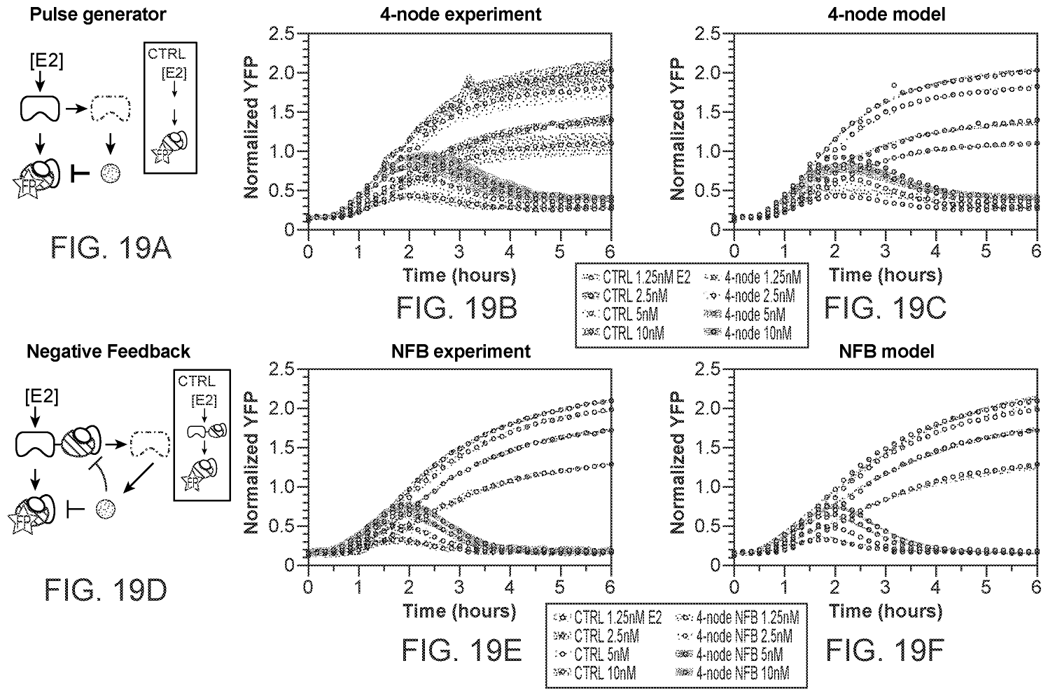

These systems were implemented using the degronLOCKR system and a few transcription factors, to obtain unable degradation, positive and negative feedback, sequestration, pulse generation, and filtering (FIG. 17, B). By leveraging the unique modularity of designer proteins these functionalities were demonstrated individually and then small circuits were layered to obtain diverse behaviors. To induce expression of the designer proteins, create transcriptional delays, and implement positive feedback three transcription factors were used. GEM and ZPM are engineered inducible transcription factors built by fusing a DNA binding domain, an inducible domain, and an activation domain (Aranda-Diaz et al., 2017). In the presence of the hormone estradiol (E2), GEM traffics into the nucleus and activates transcription at promoters with Gal4 sites like pGall. ZPM is induced by the hormone progesterone (Pg) and activates promoters with its zinc finger binding site. SynTF is a zinc finger domain bound to an activation domain and activates promoters with p43 binding sites. Positive feedback is implemented by adding an additional GEM controlled by its own promoter (SI figure reference showing how PFB works). The strength of positive feedback was tuned by modulating the basal GEM expression. Tunable degradation and negative feedback have been explored in detail in previous publications (Langan et al., 2019; Ng et al., 2019). Sequestration is implemented using the ‘decoy’ to sequester key creating a threshold the key needs to overcome before it can bind to the cage. Using these 6 parts many different signal processing units were constructed.

The core motif used here for pulse generation and filtering was a type-1 incoherent feedforward loop (I1-FFL), which can generate a pulse of gene expression (Basu et al., 2004; Mangan et al., 2003) and accelerate the response time (Mangan et al., 2006). I1-FFLs have also been used to implement band-pass filters, fold-change detection, biosensing and noise buffering. An I1-FFL is made up of an activator X that activates a gene Z and simultaneously its repressor, Y. It can produce a pulse of gene Z expression because the activation reaction is triggered immediately by X, while the dominating repression occurs with a delay due to the presence of the intermediate component Y. The I1-FFL architecture can also produce filtering behavior when the activating and repressing transfer functions overlap in the correct way. If the activation responds at a lower inducer concentration than the repressor then there is a window for the circuit to respond with high outputs for an intermediate range of inputs giving a low-high-low response. This range can be tuned by tuning the transfer functions of the activator and repressor.

### Material and Methods

Construction of DNA circuits. Hierarchical golden gate assembly was used to assemble plasmids for yeast strain construction based on the Yeast Toolkit standard (Lee et al., 2015). Parts were either generated via PCR or purchased as gBlocks from IDT. These parts were then assembled into transcriptional units (promoter-gene-terminator) on cassette plasmids. These cassettes were then assembled together to form multi-gene plasmids for insertion into the yeast genome. All DNA manipulations were performed with standard molecular biology techniques.

Yeast strains and growth media. The base Saccharomyces cerevisiae strain used in all experiments was BY4741 (MATa his3 Δ1 leu2 Δ0 met15 Δ0 ura3 Δ0). Overnight yeast cultures were grown in YPD medium (1% w/v bacto-yeast extract, 2% w/v bacto peptone and 2% w/v dextrose). Yeast transformation cultures and time course experiment cultures were diluted in YPD. Cultures for steady state flow cytometry experiments were diluted in SDC (0.67% w/v bacto-yeast nitrogen base without amino acids, 0.2% complete supplement mixture (MP Biomedicals), and 2% w/v dextrose). SDC agar plates with the appropriate nutrient removed (Teknova) were used for selection after transformation. Yeast transformations were performed using a standard lithium acetate procedure.

Yeast cell culture and induction. Yeast strains were streaked out from a glycerol stock on YPD plates. Individual colonies from these plates were used to inoculate YPD cultures to grow to saturation over 12-24 h.

Testing the steady state circuit response for the positive feedback network. Saturated culture was diluted 1:250 in fresh SDC and 450 μl was aliquoted into individual wells of a 2-ml V-bottom 96-well block (Corning/Costar) for a 1.5-h outgrowth at 30° C. and 900 r.p.m. in a Multitron shaker (Infors HT). During the 1.5-h outgrowth, estradiol (Sigma-Aldrich) was prepared at a 10× concentration by making an appropriate dilution into SDC. A one-to-three serial dilution was performed to create an estradiol gradient across the wells. After the 1.5-h outgrowth, 50 μl of estradiol solution was added to the 96-well block and the block was returned to the shaker for 5 h before measurement with flow cytometry.

Testing the steady state circuit response for the decoy and filter networks. Saturated culture was diluted 1:250 in fresh SDC and 400 μl was aliquoted into individual wells of a 2-ml V-bottom 96-well block (Corning/Costar) for a 1.5-h outgrowth at 30° C. and 900 r.p.m. in a Multitron shaker (Infors HT). During the 1.5-h outgrowth, Estradiol (Sigma-Aldrich) and progesterone (Fisher Scientific) were prepared at a 10× concentration by making the appropriate dilutions into SDC. One-to-three serial dilutions were performed to create estradiol and progesterone gradients. After the 1.5-h outgrowth, 50 μl of estradiol solution and 50 μl of progesterone solution was added to the 96-well block in a matrix format and the block was returned to the shaker for 5 h before measurement with flow cytometry.

Automated flow cytometry and continuous culture system. Hardware. To perform the time course experiments, an existing experimental platform that performs automated induction, dilution, and sampling at regular intervals was used (Harrigan et al., 2018; Ng et al., 2019). Yeast cultures were grown in 50-ml conical tubes (Falcon) that were held in 8 custom temperature-controlled, magnetically stirred chambers. Sampling frequency and dilution volume were selected to avoid saturation of culture on the basis of the duration of the experiment. For experiments longer than 6 h, a sampling frequency of 25 min and a dilution volume of 2.5 ml were used. For experiments shorter than six hours, continuous culturing was not performed. Instead, a single induction at 0 h was performed by extracting 2 ml of culture and replenishing with fresh medium and hormone. A sampling frequency of 10 min was used.

Yeast culture. Saturated culture was diluted 1:100 into fresh YPD. Cultures were grown for 1 h in glass tubes at 30° C. and 250 r.p.m. in an Innova 44 shaker (New Brunswick). Cultures were then diluted to an optical density at 600 nm of 0.01 in fresh YPD, and aliquoted into individual 50-ml conical tubes (Falcon) at a total volume of 30 ml YPD. Another 45 minute outgrowth was performed in bioreactors with magnetically controlled stir bars at 30° C. All YPD medium was supplemented with 5,000 U/ml penicillin streptomycin (Thermo Fisher).

Induction with estradiol for testing pulse generation over 6 hours. A 1× concentration was determined by the highest-desired hormone concentration at which to test strains (10 nM estradiol). YPD medium was prepared at two concentrations: (1) 15× estradiol concentration and (2) no hormone. After a 45 minute outgrowth in bioreactors, induction was performed by extracting 2 ml from all cultures and replenishing with various ratios of concentrations (1) and (2) to achieve the desired concentrations. Sampling proceeded without dilution.

Induction with estradiol for testing pulse generation over 12-15 hours. A 1× concentration was determined by the highest-desired hormone concentration at which to test strains (10 nM estradiol). A solution of hormone and YPD medium was created at a 12× concentration to bring pre-induced cultures to a desired concentration in one sampling period. YPD medium was prepared at 3 concentrations of hormone: (1) 12× estradiol (2) 1× estradiol (3) no hormone. After a 45 minute outgrowth in bioreactors, the first induction was performed by extracting 2.5 ml from all cultures and replenishing with the 12× stock. All sampling periods following the induction time point included sending a sample to the cytometer for measurement, extracting 2.5 ml from all cultures and replenishing cultures with a mixture of concentrations (2) and (3) to maintain the desired hormone concentration.

Flow cytometry. Analysis of the expression of fluorescent protein reporters was performed with a BD LSRII flow cytometer (BD Biosciences) equipped with a high-throughput sampler. For steady-state measurements, cultures were diluted in TE before running through the instrument to obtain an acceptable density of cells. YFP (Venus) fluorescence was measured using the FITC. For steady-state measurements, 5,000-10,000 events were collected per sample. For dynamic measurements, the first 750 events of the sample were discarded, and 2,000-10,000 events were collected per sample. Fluorescence values were calculated using the height measurement for the appropriate channel and normalized to cell size by dividing by side scatter (SSC-H). All analysis of flow cytometry data was performed in Python 2.7 using the package FlowCytometryTools and custom scripts.

### Results

### Construction and Characterization of Simple Networks

Three simple networks were built and tested that act as the building blocks for more complex networks: positive feedback, sequestration, and a 3 node I1-FFL.

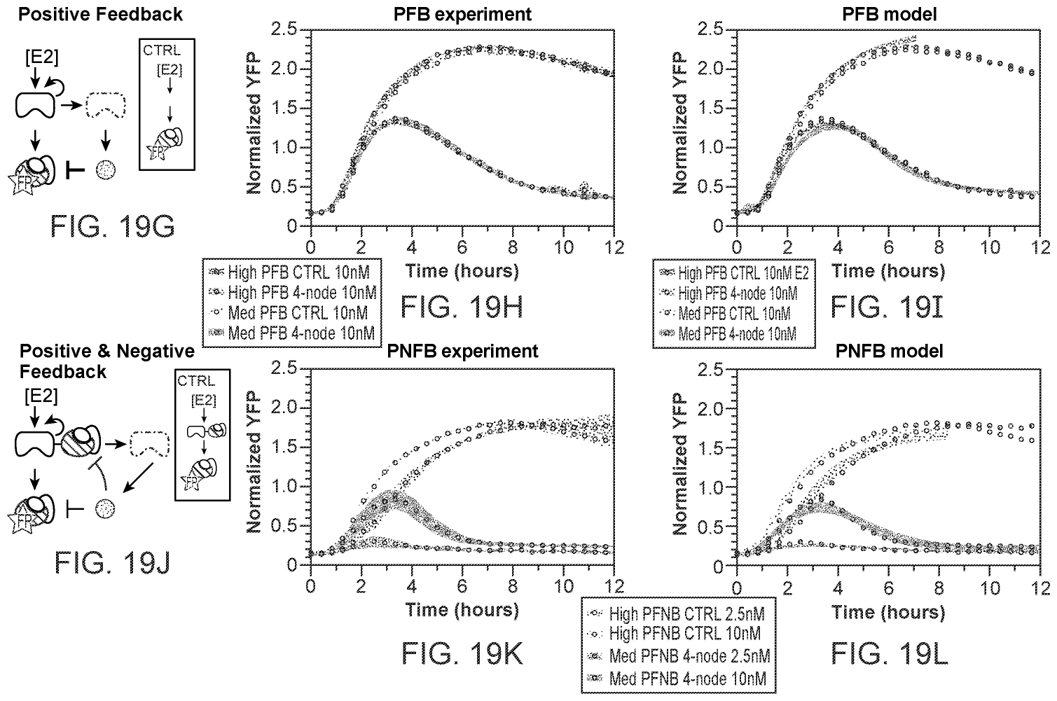

Positive feedback amplifies and shifts the response. It was first sought to demonstrate positive feedback with GEM, a hormone inducible synthetic transcription factor. The positive feedback network consists of two copies of GEM (orange) activating YFP (yellow) (FIG. 18, A). One copy of GEM is under a constitutive promoter (pPAB1 ‘high’, pRNR2 ‘medium’, or pREV1 ‘low’) and the other is under pGall which is activated by GEM. The constitutive promoter allowed the setting of a basal GEM expression level. When the system is induced with estradiol, the basal GEM is activated, shuttles into the nucleus, and activates its own expression creating a positive feedback loop. The positive feedback network in yeast cells was tested by integrating the circuit into the genome. After 5 hours of induction with estradiol (0-100 nM E2) steady state fluorescence was measured on a cytometer and it was found that positive feedback amplifies the response and increases its sensitivity (FIG. 18, A). Increasing the promoter strength on the constitutive GEM shifts the positive feedback response.

Sequestration by decoy shifts the key/cage transfer function. It was next sought to shift the transfer function of the cage/key relationship in order to make filtering possible. Sequestration is known to be able to shift a transfer function. The decoy, a truncated version of the cage with no latch, that can bind to the key and sequester it, blocking it from binding to the cage, was designed. This decoy mediated sequestration was tested by building a circuit with the decoy (dark blue) under pGall such that it is activated by estradiol through GEM (orange), the key (green) under pZ3 such that it is activated by progesterone through ZPM (purple), and YFP-cage (yellow/light blue) under constitutive expression (FIG. 18, B). After induction with a range of estradiol (0 nM, 2.5 nM, 5 nM, and 10 nM E2) and progesterone (0-100 nM Pg) for 5 hours YFP fluorescence was measured on a cytometer and it was found that the key/cage transfer function (FIG. 18, B) is shifted to the right by increasing the concentration of decoy through progesterone. This allows tuning of the key/cage relationship to optimize a filter circuit.

The 3-node II-FFL exhibits weak pulse generation. The exploration of the I1-FFL began by building the original 3-node design. The implementation uses GEM (orange) as the input node, key (green) as the intermediate node, and YFP-cage (yellow/light blue) as the output node (FIG. 18, C). GEM activates key and YFP-cage and as key is transcribed it causes the degradation of YFP-cage. This circuit was integrated into the yeast genome and its functionality was analyzed by inducing with 4 different estradiol concentrations (1.25 nM, 2.5 nM, 5 nM, and 10 nM E2) and measuring YFP fluorescence dynamics using flow cytometry (FIG. 18, C). The circuit without key, just GEM activating YFP-cage, was included as a control. A previously described automated flow cytometry robot was used to collect fluorescence data every 10 minutes for 8×30 mL cultures over 6 hours. The 3-node IFFL exhibits very slight pulsing behavior.

In order to quantify the pulsing characteristics of the 3-node I1-FFL the normalized reduction from pulse peak at steady state was calculated. This value is equal to ([average of the final 3 time points]−[average of the first 3 time points])/([maximum value]−[average of the first 3 time points]). For the 10 nM E2 condition the normalized reduction from the pulse peak at steady state was 80%. This means the steady state is 80% of the pulse peak.

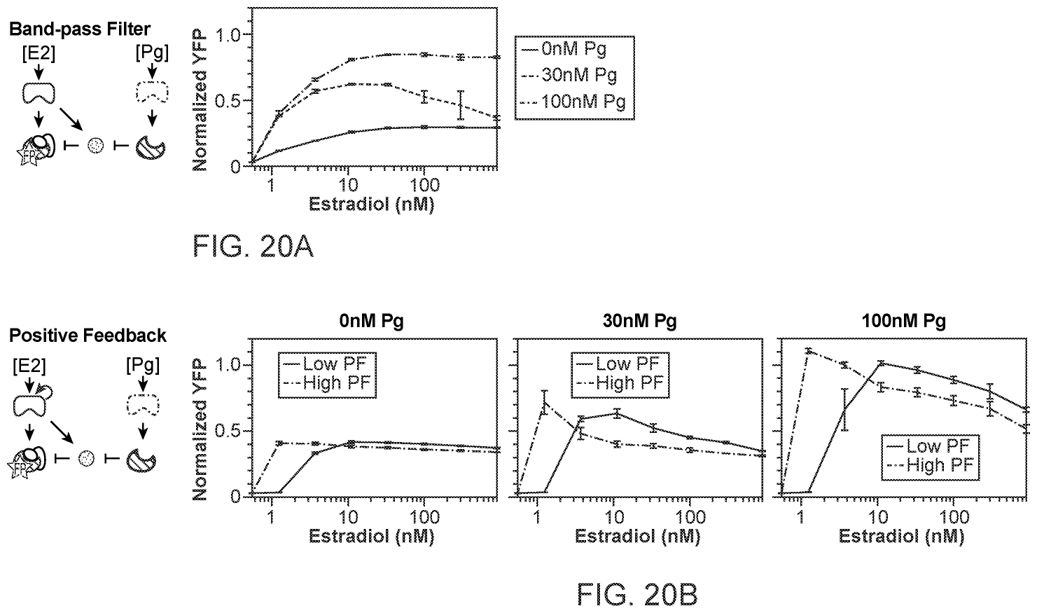

Four node pulse generators with positive and negative feedback. It was hypothesized that the reason the 3-node I1-FFL was not pulsing sufficiently was because there was not a large enough delay in the repression arm. If the activated degradation caused by key binding is much faster than transcription and translation then the activating and repressing arms of the 3-node I1-FFL may be on the same timescale. In order to improve the pulsing behavior of the pulse generator, a transcriptional delay was added by inserting a constitutive transcription factor, SynTF (magenta), into the circuit. In the 4-node I1-FFL GEM activates YFP-cage as well as SynTF. SynTF then activates key which interacts with YFP-cage to activate its degradation (FIG. 19, A). This circuit was integrated into the yeast genome and its functionality was analyzed by inducing with 4 different estradiol concentrations (1.25 nM, 2.5 nM, 5 nM, and 10 nM E2) and measuring YFP fluorescence dynamics using automated flow cytometry (FIG. 19, B). The circuit without key or SynTF, just GEM activating YFP-cage, was included as a control. It was found that the steady state was lower when a fourth node was added but the pulse heights were similar between the 3 and 4-node circuits. This indicates that the source of the improvement is from stronger key activity, likely due to amplification of key caused by the transcriptional cascade. For the 10 nM E2 condition the normalized reduction from the pulse peak at steady state was 33% which is a large improvement in pulsing behavior over the 3-node I1-FFL.

Negative feedback reduces the steady state. To further reduce the steady state expression it was decided to add negative feedback to the 4-node I1-FFL. Negative feedback was built into the circuit by fusing the cage to GEM (FIG. 19, D). In this way, key will now repress both the output of the circuit (YFP-cage) as well as the input (GEM-cage) and reduce the overall output after key is expressed. This circuit was integrated into the yeast genome and its functionality was analyzed by inducing with 4 different estradiol concentrations (1.25 nM, 2.5 nM, 5 nM, and 10 nM E2) and measuring YFP fluorescence dynamics using automated flow cytometry (FIG. 19, E). The circuit without key or SynTF, just GEM-cage activating YFP-cage, was included as a control. As expected, it was found that both the steady state and the pulse height were lower when negative feedback was added. For the 10 nM E2 condition, the normalized reduction from the pulse peak at steady state was 3%.

Positive feedback increases pulse height and duration. To increase the pulse height, it was decided to add positive feedback to the 4-node I1-FFL. Positive feedback was added to the circuit by creating a positive feedback loop on GEM as in FIG. 18, A. For this circuit, two constitutive GEM promoters were tested: high (pPAB1) and medium (pRNR2). This circuit was integrated into the yeast genome and its functionality was analyzed by inducing with one estradiol concentration (10 nM E2) and measuring YFP fluorescence dynamics using automated flow cytometry (FIG. 19, E). The circuit without key or SynTF, just GEM with positive feedback activating YFP-cage, was included as a control. When adding positive feedback it was found that the pulse duration was lengthened so the time course experiments were performed over 12 hours using a dilution protocol. The dilution protocol was necessary to keep the cultures dilute enough to run through the cytometer after 6 hours. The dilution protocol has been previously used and validated (ref feedback paper). This means the data in FIGS. 19, H and K cannot be directly compared to 19, B and E. The original 4-node I1-FFL and 4-node I1-FFL with negative feedback were included using a 12 hour dilution protocol. For the 10 nM E2 condition, the normalized reduction from the pulse peak at steady state was 19% for the high promoter and 16% for the medium promoter. It was found that the pulse height and pulse duration were both increased when adding positive feedback. Additionally, the positive feedback circuit becomes insensitive to changes in input concentration or constitutive promoter strength after a certain input similarly to positive feedback alone (FIG. 19, A). This could be advantageous when designing circuits that need to be indifferent to variation in input concentration.

Positive and negative feedback. Finally, it was decided to try layering both positive and negative feedback on the 4-node I1-FFL. The two changes from the previous sections were combined by adding positive feedback on GEM-cage (FIG. 19, J). This circuit was integrated into the yeast genome and its functionality was analyzed by inducing with 2 different estradiol concentrations (2.5 nM and 10 nM E2) and measuring YFP fluorescence dynamics using automated flow cytometry over 12 hours with dilution (FIG. 19, K). The circuit without key or SynTF, just GEM-cage with positive feedback activating YFP-cage, was included as a control. For the 10 nM E2 condition the normalized reduction from the pulse peak at steady state was 13%. As expected, an increase in pulse height over negative feedback alone and a reduction in steady state fluorescence were observed.

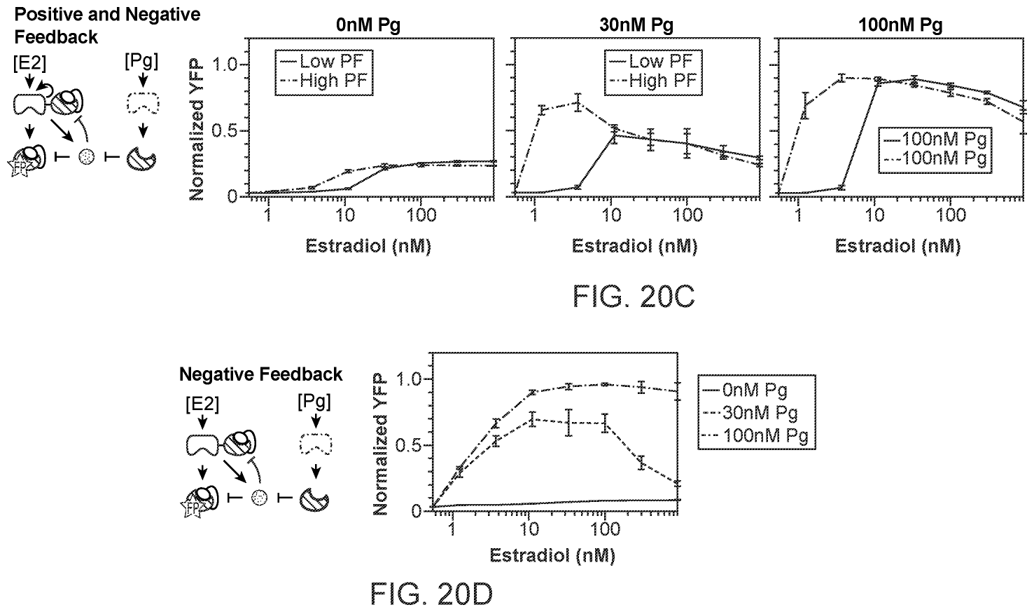

Concentration filters with positive and negative feedback. It was next sought to build a biological concentration filter by combining the 3-node I1-FFL and sequestration. A 3-node I1-FFL can act as a filter under certain conditions. The I1-FFL based filter is a combination of an activating arm and repressing arm where the activating arm is sensitive to lower inputs than the repressing arm such that there is a window where the circuit is activated but not yet repressed. For the basic 3-node I1-FFL built here there is no window between the activating and repressing arms. Decoy mediated sequestration was used to create this window by shifting the transfer function of the repression to the right. Thus, the original filter circuit is made up of the 3-node I1-FFL plus two sequestration nodes: ZPM (purple) activating the decoy (dark blue) (FIG. 20, A). This circuit was integrated into the yeast genome and its functionality was analyzed by inducing with a range of estradiol (0-900 nM E2) and 3 different progesterone concentrations (0 nM, 30 nM, and 100 nM Pg). Incubation occurred for 5 hours after induction and steady state fluorescence was measured using a flow cytometer (FIG. 20, A). The three concentrations of progesterone represent two saturation regimes and one functional regime. The 0 nM Pg represents the no decoy state which is an approximation of the 3-node I1-FFL alone. This condition did not exhibit any pulsing behavior. The 100 nM Pg represents the system flooded with decoy and approximates a state with little functional key where GEM is free to activate YFP-cage without significant repression. The 30 nM Pg is the functional filter state where high YFP output was observed for some moderate estradiol input range but low YFP for low and high estradiol.

Negative feedback reduces the high estradiol output. In order to reduce the high estradiol YFP expression and make the filter more filter-like, it was sought to add negative feedback to the filter circuit. Negative feedback was added by fusing cage to GEM in the same way it was added for the 4-node I1-FFL. Now when GEM activates key, the key will repress both GEM-cage and YFP-cage (FIG. 20, B). This circuit was integrated into the yeast genome and its functionality was analyzed by inducing with a range of estradiol (0-900 nM E2) and 3 different progesterone concentrations (0 nM, 30 nM, and 100 nM Pg). Incubation occurred for 5 hours after induction and steady state fluorescence was measured using a flow cytometer (FIG. 20, B). It was found that the 30 nM Pg condition for the filter with negative feedback does show reduced expression at high estradiol concentrations and behaves more filter-like.

Positive Feedback enables filter peak shifting. One of the key properties of GEM positive feedback is the ability to shift the circuit sensitivity using the constitutive promoter strength. It was decided to use positive feedback in the filter to shift the filter peak by adding positive feedback onto GEM with two different promoter strengths (pPAB1 ‘high’ or pREV1 ‘low’). This circuit was integrated into the yeast genome and its functionality was analyzed by inducing with a range of estradiol (0-900 nM E2) and 3 different progesterone concentrations (0 nM, 30 nM, and 100 nM Pg). Incubation occurred for 5 hours after induction and steady state fluorescence was measured using a flow cytometer (FIG. 20, C). As expected, the filter peak could be shifted using positive feedback, but the key had a hard time overcoming the positive feedback and the filter does not repress very well at high estradiol concentrations.

Positive and negative feedback. It was hoped to improve the high estradiol aspect of the positive feedback filter by adding negative feedback. This circuit uses GEM-cage with positive feedback as the input node (FIG. 20, D) with two promoter strengths (pPAB1 ‘high’ or pREV1 ‘low’). This circuit was integrated into the yeast genome and its functionality was analyzed by inducing with a range of estradiol (0-900 nM E2) and 3 different progesterone concentrations (0 nM, 30 nM, and 100 nM Pg). Incubation occurred for 5 hours after induction and steady state fluorescence was measured using a flow cytometer (FIG. 20, D). Minor improvements were observed in the circuit's ability to repress at high estradiol concentrations.

In this work, two functionalities of the I1-FFL motif have been explored: pulse generation and filtering. First, three simple networks that would help build and optimize the basic pulse generator and filter were assessed: positive feedback, sequestration, and a 3-node I1-FFL. To achieve pulse generation, a transcriptional delay was added to the 3-node I1-FFL turning it into a 4-node I1-FFL. In order to implement filtering the 3-node I1-FFL was combined with sequestration. The modular design makes it easy to layer feedback into circuits to tune the time response and steady state expression. By adding negative feedback onto the input in the 4-node I1-FFL the final expression after the pulse in a pulse generator or steady state expression at high levels of input in a filter can be decreased. Positive feedback was also used to amplify and shift the response. Positive feedback is known for its ability to amplify and increase the sensitivity of gene expression. Integrating positive feedback into the I1-FFLs provides more control over the pulse length and height and at which concentrations of inducer the filter activates.

The modularity of the designer proteins allow them to be inserted into any system of interest. One can also exchange parts within the network to alter its behavior. Promoters, transcription factors, and even the designer protein parts can be swapped to change aspects of the dynamic response and steady state expression. Additionally, the work layers feedback and feedforward to achieve a more tunable response. This provides unparalleled control over the output dynamics allowing one to make any necessary changes for each unique application. Aside from the modularity mentioned above, the designer protein has a compact mechanism that will make it easier to import into a cell and connect with endogenous pathways.

Due to the inherent repression delay, an I1-FFL pulse generator can reliably produce a consistent pulse of output regardless of the duration of input over a certain threshold. Positive feedback can also make the pulse generator indifferent to changes in the magnitude of the input. These properties make a pulse generator ideal for systems with uncertain inputs or toxic outputs. Such systems could reduce off target gene editing from Cas9, improve localized effects of cellular therapeutics, mitigate toxic metabolic intermediates in a synthetic pathway, and reduce noise in gene expression from lentiviral delivery.

When combined with a sensor, a biological concentration filter can improve sensing by allowing a network to respond only to certain concentrations of input. I1-FFL signal processing has been used in biological filter designs to control pattern formation of gene expression on bacterial agar plates. A filter could be used to pattern mammalian cells to facilitate synthetic organ production or controlled differentiation of stem cells. Cell therapies could be made more specific by engineering cells that detect only a small range of antigen concentrations on a cell surface.

### REFERENCES

Aranda-Díaz, A., Mace, K., Zuleta, I., Harrigan, P., El-Samad, H., 2017. Robust Synthetic Circuits for Two-Dimensional Control of Gene Expression in Yeast. ACS Synth. Biol. 6, 545-554.
Barone, F., Dorr, F., Marasco, L. E., Mildiner, S., Patop, I. L., Sosa, S., Vattino, L. G., Vignale, F. A., Altszyler, E., Basanta, B., Carlotto, N., Gasulla, J., Giménez, M., Grande, A., Nieto Moreno, N., Bonomi, H. R., Nadra, A. D., 2017. Design and evaluation of an incoherent feed-forward loop for an arsenic biosensor based on standard iGEM parts. Synth. Biol. 2.
Basu, S., Mehreja, R., Thiberge, S., Chen, M.-T., Weiss, R., 2004. Spatiotemporal control of gene expression with pulse-generating networks. Proc. Natl. Acad. Sci. 101, 6355-6360.
Chappell, J., Westbrook, A., Verosloff, M., Lucks, J. B., 2017. Computational design of small transcription activating RNAs for versatile and dynamic gene regulation. Nat. Commun. 8, 1051.
Chen, Z., Kibler, R. D., Hunt, A., Busch, F., Pearl, J., Jia, M., VanAernum, Z. L., Wicky, B. I. M., Dods, G., Liao, H., Wilken, M. S., Ciarlo, C., Green, S., El-Samad, H., Stamatoyannopoulos, J., Wysocki, V. H., Jewett, M. C., Boyken, S. E., Baker, D., 2020. De novo design of protein logic gates. Science 368, 78-84.
Cheng, Y.-Y., Hirning, A. J., Josid, K., Bennett, M. R., 2017. The Timing of Transcriptional Regulation in Synthetic Gene Circuits. ACS Synth. Biol. 6, 1996-2002.
Entus, R., Aufderheide, B., Sauro, H. M., 2007. Design and implementation of three incoherent feed-forward motif based biological concentration sensors. Syst. Synth. Biol. 1, 119-128.
Goentoro, L., Shoval, O., Kirschner, M. W., Alon, U., 2009. The Incoherent Feedforward Loop Can Provide Fold-Change Detection in Gene Regulation. Mol. Cell 36, 894-899.
Harrigan, P., Madhani, H. D., El-Samad, H., 2018. Real-Time Genetic Compensation Defines the Dynamic Demands of Feedback Control. Cell 175, 877-886.e10.
Kaplan, S., Bren, A., Zaslaver, A., Dekel, E., Alon, U., 2008. Diverse Two-Dimensional Input Functions Control Bacterial Sugar Genes. Mol. Cell 29, 786-792.
Langan, R. A., Boyken, S. E., Ng, A. H., Samson, J. A., Dods, G., Westbrook, A. M., Nguyen, T. H., Lajoie, M. J., Chen, Z., Berger, S., Mulligan, V. K., Dueber, J. E., Novak, W. R. P., El-Samad, H., Baker, D., 2019. De novo design of bioactive protein switches. Nature 572, 205-210.
Lee, M. E., DeLoache, W. C., Cervantes, B., Dueber, J. E., 2015. A Highly Characterized Yeast Toolkit for Modular, Multipart Assembly. ACS Synth. Biol. 4, 975-986.
Mangan, S., Itzkovitz, S., Zaslaver, A., Alon, U., 2006. The Incoherent Feed-forward Loop Accelerates the Response-time of the gal System of Escherichia coli. J. Mol. Biol. 356, 1073-1081.
Mangan, S., Zaslaver, A., Alon, U., 2003. The Coherent Feedforward Loop Serves as a Sign-sensitive Delay Element in Transcription Networks. J. Mol. Biol. 334, 197-204.
Milo, R., Shen-Orr, S., Itzkovitz, S., Kashtan, N., Chklovskii, D., Alon, U., 2002. Network Motifs: Simple Building Blocks of Complex Networks. Science.
Ng, A. H., Nguyen, T. H., Gómez-Schiavon, M., Dods, G., Langan, R. A., Boyken, S. E., Samson, J. A., Waldburger, L. M., Dueber, J. E., Baker, D., El-Samad, H., 2019. Modular and tunable biological feedback control using a de novo protein switch. Nature 572, 265-269.
Osella, M., Bosia, C., Corá, D., Caselle, M., 2011. The Role of Incoherent MicroRNA-Mediated Feedforward Loops in Noise Buffering. PLOS Comput. Biol. 7, e1001101.
Shen-Orr, S. S., Milo, R., Mangan, S., Alon, U., 2002. Network motifs in the transcriptional regulation network of Escherichia coli. Nat. Genet. 31, 64-68.
Takeuchi, J., Chen, H., Hoyt, M. A., Coffino, P., 2008. Structural elements of the ubiquitin-independent proteasome degron of ornithine decarboxylase. Biochem. J. 410, 401-407.

### Examples II

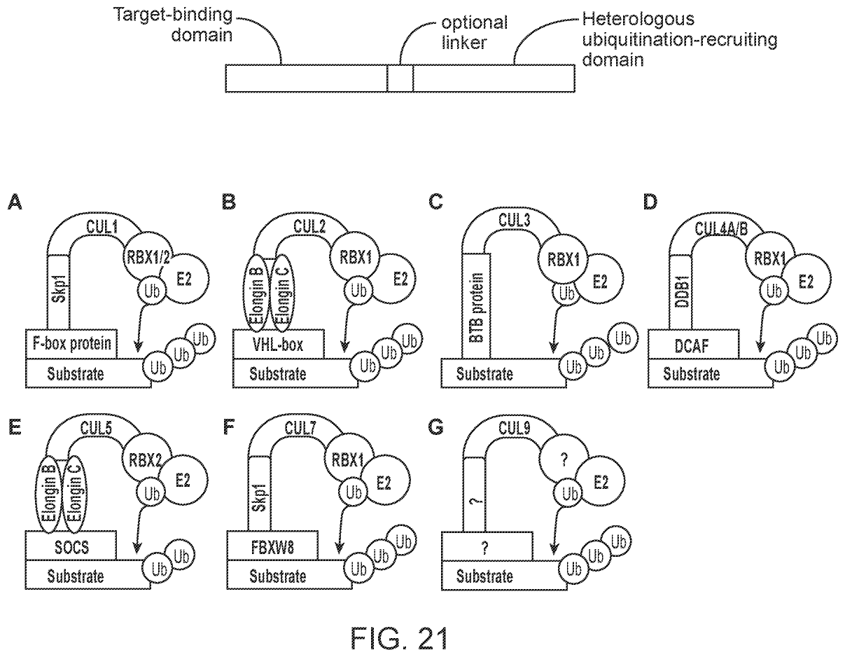

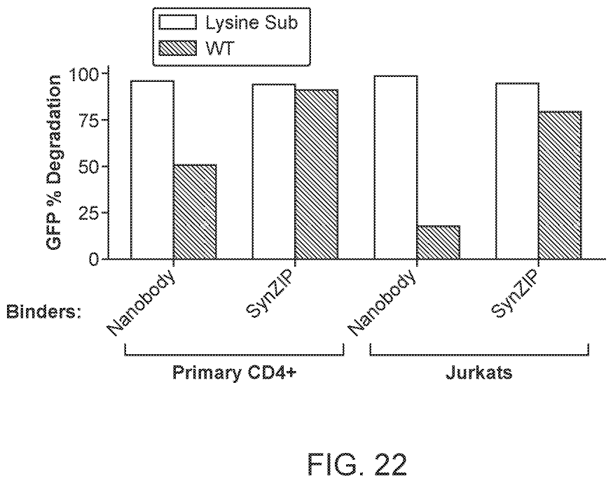

The remainder of the examples describe a degradation technology based on a protein chimera contains a protein targeting domain, an optional linker, and a protein degradation domain, e.g., a degron. This protein chimera is able to recruit the endogenous E3 ligase machinery of the cell to novel targets, triggering the ubiquitination and degradation of natural and unnatural targets. This tool is referred to as a “synthetic targetter of ubiquitination and degradation”, or “STUD” for short. A particularly potent C-terminal minimal degron motif of the sequence RRRG (Arg-Arg-Arg-Gly; also referred to as the “Bonger” motif) was used as a basis for developing this technology. In theory, this system should be amenable to a variety of degron motifs or E3 scaffold domains. The feedback circuits described herein can be implemented using STUDs in some cases. Examples of STUDS are shown in FIGS. 21 and 22.

### Example 1

### Cis-Ubiquitination can be Prevented by Substituting the Lysines in a STUD

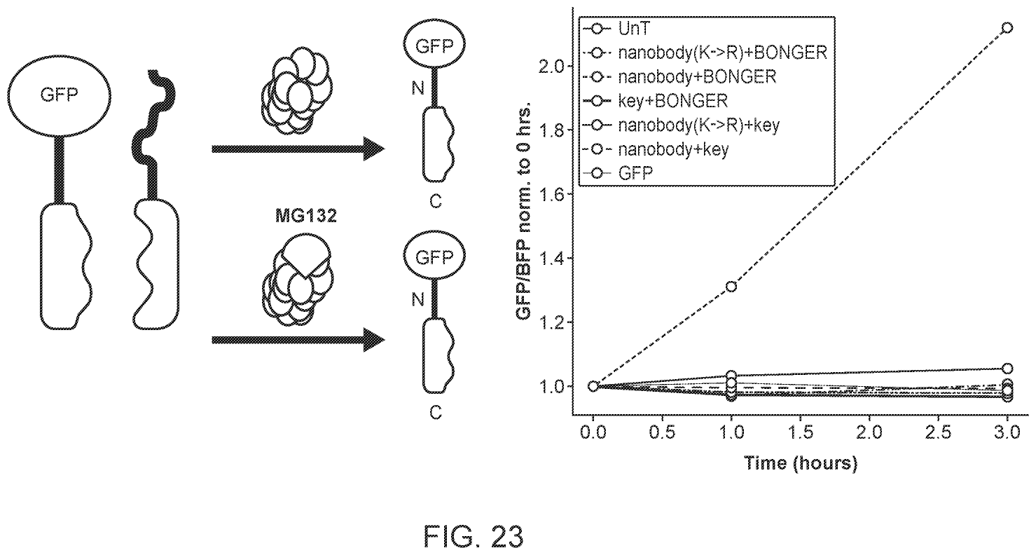

This protein degradation tool has the potential to ubiquitinate target lysines on both the target of interest (trans-ubiquitination), as well as on the tool itself (cis-ubiquitination). cis-ubiquitination may limit the effectiveness of the STUD by degrading the STUD before it has the chance to interact with its target. To solve this problem, the lysines on the protein targeting domain of the STUD were mutated to arginines (K->R), thus preventing cis-ubiquitination2. An assay was developed to test the functionality of a STUD by measuring degradation of a cystosolic GFP. The GFP was targeted for degradation using either a GFP nanobody or a SynZIP17 that was fused to the GFP. The target GFP was transduced into either Jurkat cells or primary human T cells using lentivirus and the STUD was introduced via a second lentivirus. It was observed that the lysine substitution significantly improved the activity of the GFP nanobody STUD, whereas the mutation only moderately improved the activity of the SynZIP STUD. These results are shown in FIG. 23. This trend was consistent between primary human CD4+ T cells and Jurkats. Given these results, it should be possible to use the number of lysines on the STUD as a strategy for tuning the activity of the STUD, where more mutated lysines increases the activity of the STUD.

### Example 2

### STUD-Induced Degradation is Mediated Via the Proteasome

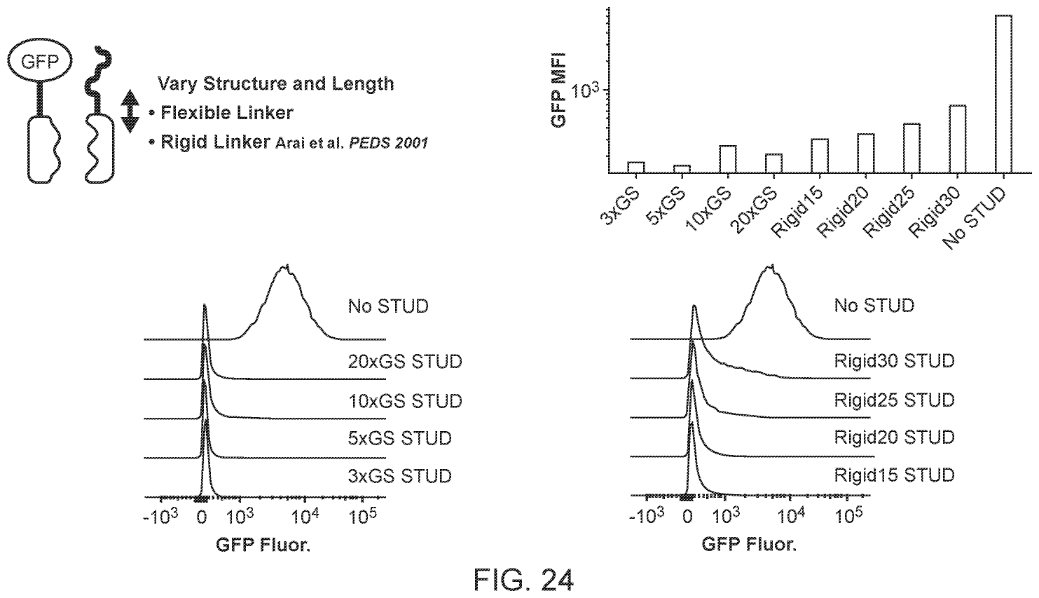

The mechanism of how the STUD reduces GFP was explored. Primary human CD4+ T cells expressing the GFP nanobody STUD were fed with the MG132 proteasome inhibitor and the change in fluorescence was measured over time. These results are shown in FIG. 24. Cells expressing a functional STUD should display an increase in fluorescence over time as the proteasome inhibitor took effect. After three hours of exposure to the drug, it was observed that only the cells expressing the functional nanobody STUD (nanobody(K->R)+Bonger) displayed an increase in GFP fluorescence. This indicates that the observed reduction in GFP is mediated by degradation via the proteasome rather than a mechanism associated with the protein-protein interaction alone.

### Example 3

### STUD Activity can be Optimized Using a Linker

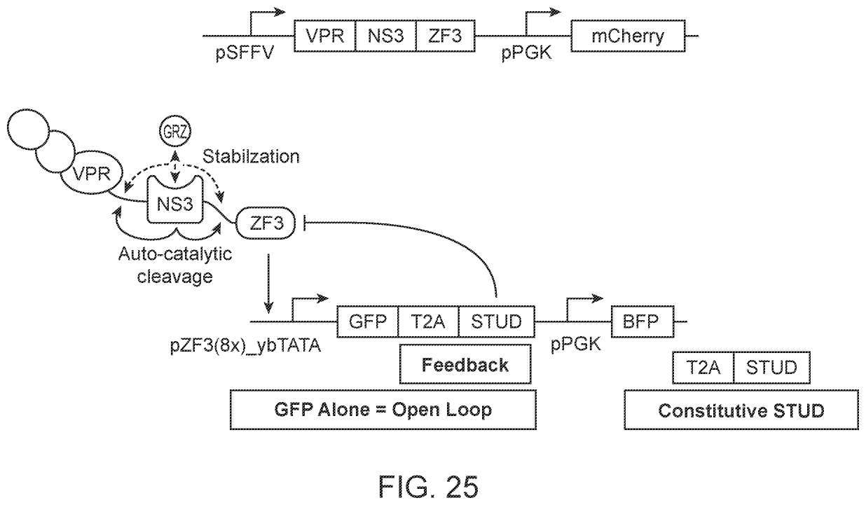

The STUD was optimized by screening multiple lengths of two different classes of linkers. In these constructs, the linker was added between a SynZIP protein binding domain and the Bonger degron. It was hypothesized that a flexible Gly-Ser linker may facilitate target degradation by increasing the accessibility of the E3 ligase to reach target lysine residues on the surface of the target protein, whereas a rigid helical linker may increase the distance between the E3 ligase and target lysines and reduce degradation. These experiments used the SynZIP STUD that targets cytosolic GFP-SZ17 as described above. Four lengths of linker for both the flexible and rigid linker. The flexible linker generally performed better than the rigid linker, with little variation in degradation efficiency observed within the different flexible linker lengths (FIG. 25). However among the flexible linkers the 5×GS performed the best. This STUD (with the SynZIP(K->R), optimized linker and C-terminal RRRG (SEQ ID NO:1), or SynZIP18(K->R)-5×GS-RRRG; SEQ ID NO:2) is referred to as the “soluble stud” and used in the following experiments.

### Example 4

### Transcription Factors can be Targeted

Lysine substitution and linker length/type optimization served as a framework for optimizing future STUD iterations that use other protein targeting domains and/or degradation domains, e.g., degrons. Depending on the application, different synthetic protein targeting domains may be more suitable, and it is also possible to utilize endogenous protein targeting domains that bind to or interact with an endogenous protein without the need for modification of the endogenous protein. Furthermore, different degrons may be utilized to vary the conditions under which the STUD is active, or confine the activity of the STUD to different compartments of the cell where the degron is active.

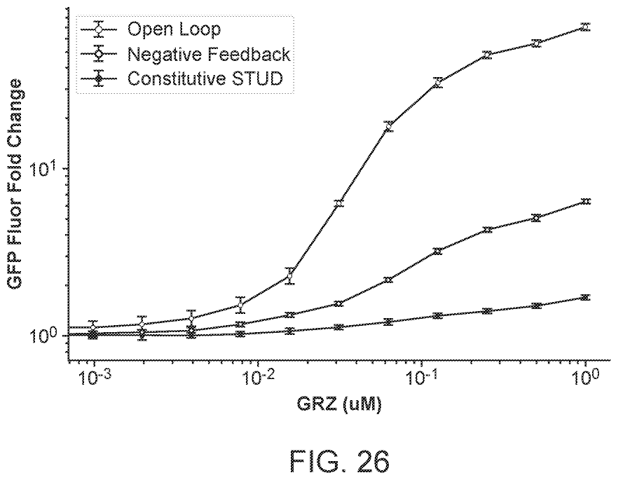

A transcription factor was targeted for degradation using the soluble STUD described above. Modulating a transcription factor allows one to affecting the output of a functional protein. Thee experiments were done using a previously developed grazoprevir (GRZ) drug-inducible zinc-finger transcription factor system (VPR-NS3-ZF3). To induce degradation of this transcription factor SynZIP17 to the C-terminus of this protein. Degradation of the TF was measured by observing changes in GFP reporter output driven by the pZF3(8x)ybTATA promoter. Two different methods were used for STUD expression: constitutive STUD expression, or inducible STUD expression, which should drive negative feedback in the system (FIG. 26).

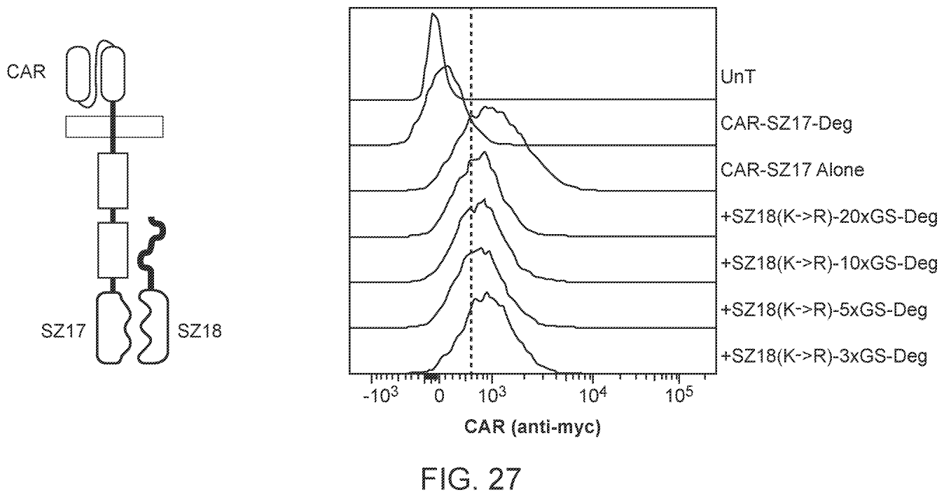

The dose responses of the three circuit variants were compared to assess the functionality of the STUD. It was found that constitutive expression of the STUD abolished nearly all output from the pZF3, whereas feedback expression of the STUD generated an intermediate dose response (FIG. 27). This demonstrates that the soluble STUD can not only degrade functional proteins in the cell, but also be used as a powerful tool for building genetic circuits.

1. A cell comprising a molecular circuit comprising:
(a) a nucleic acid encoding a target protein that comprises a regulatory motif that is not native to the target protein;
(b) a nucleic acid encoding an inactivating protein, wherein the inactivating protein binds to the regulatory motif and inactivates the target protein; and
(c) an actuating protein that, in response to a first external stimulus, independently activates expression the target protein of (a) and the inactivating protein of (b).

2. The cell of claim 1, wherein the actuating protein independently activates transcription of the nucleic acid of (a) and the nucleic acid of (b) in response to the exogenous stimulus.

3. The cell of claim 1, wherein the exogenous stimulus:
(i) binds to the actuating protein or to a protein that is upstream of the actuating protein, wherein binding activates the actuating protein,
(ii) induces expression of the actuating protein, or
(iii) binds to a transmembrane protein on the outside of the cell, wherein binding initiates a signal transduction event that results in activation of the actuating protein.

4. The cell of claim 1, wherein activation of expression of the inactivating protein by the actuating protein is delayed relative to activation of expression of the target protein by the actuating protein.

5. The cell of claim 4, wherein the actuating protein: (i) directly activates expression of the target protein and (ii) indirectly activates expression of the target protein.

6. The cell of claim 1, further comprising
(d) a controller protein that controls the interaction between the inactivating protein of (b) and the target protein of (a).

7. The cell of claim 6, wherein the controller protein controls the interaction between the inactivating protein of (b) and the target protein of (a) by binding to or inactivating the inactivating protein or by blocking the interaction between the inactivating protein and the target protein.

8. The cell of claim 6, wherein expression and/or activity of the controller protein is modulated by a second exogenous stimulus.

9. The cell of claim 1, wherein the target protein is cell surface receptor, an intracellular kinase or engineered transcription factor.

10. The cell of claim 1, wherein the actuating protein is transcription factor.

11. The cell of claim 1, wherein inactivating protein induces degradation of the target protein.

12. The cell of claim 11, wherein:
(i) the target protein comprises a caged degron and the inactivating protein comprises a molecular key that exposes the degron, thereby causing degradation of the target protein;
(ii) the inactivating protein comprises a degron and binding of the inactivating protein to the targeting protein causes degradation of the target protein in trans;
(iii) the target protein contains an internal degron and a protease cleavage site, and the inactivating protein is a protease that cleaves at the protease cleavage site and activates the degron, thereby causing degradation of the target protein.

13. The cell of claim 1, wherein the inactivating protein comprises a sub-cellular targeting domain, and binding of inactivating protein to the target protein sequesters the target protein.

14. The cell of claim 1, wherein the inactivating protein inhibits the target protein in a dominant negative manner.

15. The cell of claim 1, wherein the cell is an immune cell.

16. The cell of claim 15, wherein the cell is a T cell, Natural Killer cell or macrophage.

17. The cell of claim 15, wherein the cell is a stem cell.

18. A method comprising:
exposing a cell of claim 1 to the first external stimulus, thereby activating expression the target protein of (a) and the inactivating protein of (b).

19. The method of claim 18, wherein the cell further comprises a controller protein that controls the interaction between the inactivating protein of (b) and the target protein of (a), wherein expression and/or activity of the controller protein is modulated by a second exogenous stimulus, and the method further comprises:
exposing the cell to the second external stimulus.

20. The method of claim 18, wherein the method is done in vivo, ex vivo, or in vitro.


SEQUENCE LISTING (2 sequences)

SEQ ID NO: 1 - protein, 4 aa, Artificial sequence
  Synthetic peptide
RRRG

SEQ ID NO: 2 - protein, 6 aa, Artificial sequence
  synthetic peptide
GSRRRG